(19) United States
(12) Patent Application Publication
(10) Pub. No.: US 2025/0205453 A1
(43) Pub. Date: Jun. 26, 2025

(54) DRUG ELUTING APPARATUS AND METHOD OF DELIVERING DRUG

(71) Applicant: TERUMO MEDICAL CORPORATION, Somerset, NJ (US)

(72) Inventors: Sarah Gettinger, West Chester, PA (US); Seiichi Otsubo, Newark, DE (US)

(73) Assignee: TERUMO MEDICAL CORPORATION, Somerset, NJ (US)

(21) Appl. No.: 18/851,793

(22) PCT Filed: Mar. 20, 2023
(86) PCT No.: PCT/US2023/015684

§ 371 Date: Sep. 27, 2024

Related U.S. Application Data
(60) Provisional application No. 63/325,983, filed on Mar. 31, 2022.

(51) Int. Cl. A61M 25/01; A61M 25/10

(52) U.S. Cl. CPC A61M 25/0119; A61M 25/1009

(57) ABSTRACT

A drug eluting apparatus includes: a handle comprising a drug port configured to receive a drug; a shaft connected to the handle, the shaft including: a drug lumen configured to receive the drug from the drug port, and a guidewire tube configured to receive a guidewire and allow the guidewire to extend out of a distal portion of the shaft; a balloon attached to the distal portion of the shaft and configured to receive the drug from the drug lumen, the balloon comprising holes configured to allow the drug to exit the balloon; an anchor connected to a distal portion of the balloon; an elastically deformable wire having a distal end connected to the distal portion of the balloon, and a proximal end connected to the guidewire tube; and a pull wire connected to the anchor.

### BACKGROUND

The present application relates generally to drug eluting apparatuses for delivering a drug, such as a drug that includes an anti-proliferative agent, into a blood vessel. More specifically, the present application relates to drug eluting apparatuses that allow for compression of a balloon through which the drug is delivered, so as to deliver an additional portion of the drug into the vessel and reduce the amount of drug that remains in the balloon.

Vascular diseases may result in stenosis and restenosis of vessels, in which the buildup of plaque on the walls of the vessels results in a narrowing of the vessels, and are detrimental to health if the vessel is not treated by reopening the vessel through a treatment such as angioplasty. During angioplasty, an expansion device such as a balloon is inserted into a vessel and advanced to the site of the vessel narrowing, and is then expanded in a radially outward direction against the vessel walls to expand the vessel. Such a device may also deliver a drug to the vessel, for example, through the balloon.

### SUMMARY

In known drug eluting apparatuses, following expansion of the balloon, a significant portion of the drug remains inside the balloon and is wasted.

According to certain embodiments, after the balloon is expanded in the vessel, the balloon is compressed to deliver an additional portion of the drug into the vessel, so that a reduced amount of drug remains in the balloon.

In some embodiments, a drug eluting apparatus includes a handle including a drug port configured to receive a drug. The drug eluting apparatus also includes a shaft connected to the handle. The shaft includes a drug lumen configured to receive the drug from the drug port, and a guidewire tube configured to receive a guidewire and allow the guidewire to extend out of a distal portion of the shaft. The drug eluting apparatus also includes a balloon attached to the distal portion of the shaft and configured to receive the drug from the drug lumen, the balloon including a plurality of holes configured to allow the drug to exit the balloon. The drug eluting apparatus also includes an anchor connected to a distal portion of the balloon. The drug eluting apparatus also includes an elastically deformable wire having a distal end connected to the distal portion of the balloon, and a proximal end connected to the guidewire tube. The drug eluting apparatus also includes a pull wire connected to the anchor. The handle includes an actuator that is actuatable by a user to pull the pull wire and thereby cause the balloon to change from a longitudinally expanded state to a longitudinally compressed state. After the pull wire is pulled and the balloon is compressed, the elastically deformable wire causes the balloon to return to the longitudinally expanded state.

In some embodiments, a drug eluting apparatus includes a handle including a drug port configured to receive a drug. The drug eluting apparatus also includes a shaft connected to the handle, the shaft including a drug lumen configured to receive the drug from the drug port, and a guidewire tube configured to receive a guidewire and allow the guidewire to extend out of a distal portion of the shaft. The drug eluting apparatus also includes a carrier tube connected or connectable to the handle, the carrier tube surrounding the shaft. The drug eluting apparatus also includes a balloon attached to a distal portion of the shaft and configured to receive the drug from the drug lumen, the balloon including a plurality of holes configured to allow the drug to exit the balloon. The handle includes an actuator that is actuatable by a user to pull the shaft and thereby pull the balloon into the carrier tube and cause the balloon to change from a radially expanded state to a radially compressed state.

In some embodiments, a drug eluting apparatus includes a handle including a drug port configured to receive a drug. The drug eluting apparatus also includes a shaft connected to the handle. The shaft includes a drug lumen configured to receive the drug from the drug port, and a guidewire tube configured to receive a guidewire and allow the guidewire to extend out of a distal portion of the shaft. The drug eluting apparatus also includes a balloon attached to the distal portion of the shaft and configured to receive the drug from the drug lumen, the balloon including a plurality of holes configured to allow the drug to exit the balloon. The drug eluting apparatus also includes an anchor connected to a distal portion of the balloon. The drug eluting apparatus also includes a pull wire connected to the anchor. The handle includes an actuator that is actuatable by a user to pull the pull wire and thereby cause the balloon to change from a longitudinally expanded state to a longitudinally compressed state. The longitudinally compressed state of the balloon is an inverted state.

In some embodiments, a drug eluting apparatus includes a handle including a drug port configured to receive a drug. The drug eluting apparatus also includes a first shaft connected to the handle, the first shaft including a drug lumen configured to receive the drug from the drug port. The drug eluting apparatus also includes a second shaft connected to the handle, the second shaft configured to extend through and move independently of the first shaft, and the second shaft configured to receive a guidewire and allow the guidewire to extend out of a distal portion of the second shaft. The drug eluting apparatus also includes a balloon including a proximal portion connected to a distal portion of the first shaft, and a distal portion connected to a distal portion of the second shaft. The balloon is configured to receive the drug from the drug lumen, and the balloon includes a plurality of holes configured to allow the drug to exit the balloon. The actuator is actuatable by a user to rotate the second shaft relative to the first shaft and thereby cause the balloon to change from a radially expanded state to a radially compressed state.

In some embodiments, a method of delivering a drug into a vessel includes providing a drug eluting apparatus that includes a handle including a drug port configured to receive a drug. The drug eluting apparatus also includes a shaft connected to the handle. The shaft includes a drug lumen configured to receive the drug from the drug port, and a guidewire tube configured to receive a guidewire and allow the guidewire to extend out of a distal portion of the shaft. The drug eluting apparatus also includes a balloon attached to the distal portion of the shaft and configured to receive the drug from the drug lumen, the balloon including a plurality of holes configured to allow the drug to exit the balloon. The drug eluting apparatus also includes an anchor connected to a distal portion of the balloon. The drug eluting apparatus also includes an elastically deformable wire having a distal end connected to the distal portion of the balloon, and a proximal end connected to the guidewire tube. The drug eluting apparatus also includes a pull wire connected to the anchor. The handle includes an actuator that is actuatable by a user to pull the pull wire and thereby cause the balloon to change from a longitudinally expanded state to a longitudinally compressed state. After the pull wire is pulled and the balloon is compressed, the elastically deformable wire causes the balloon to return to the longitudinally expanded state. The method also includes inserting the distal portion of the shaft into a vessel. The method also includes providing the drug to the balloon via the drug lumen so as to inflate the balloon and deliver the drug into the vessel via the plurality of holes of the balloon. The method also includes compressing the balloon by using the actuator to pull the pull wire, thereby causing the balloon to change from the longitudinally expanded state to the longitudinally compressed state and expelling at least a portion of the drug remaining in the balloon. The method also includes allowing the elastically deformable wire to cause the balloon to return to the longitudinally expanded state.

In some embodiments, a method of delivering a drug into a vessel includes providing a drug eluting apparatus that includes a handle including a drug port configured to receive a drug. The drug eluting apparatus also includes a shaft connected to the handle, the shaft including a drug lumen configured to receive the drug from the drug port, and a guidewire tube configured to receive a guidewire and allow the guidewire to extend out of a distal portion of the shaft. The drug eluting apparatus also includes a carrier tube connected to the handle, the carrier tube surrounding the shaft. The carrier tube has a length that is equal to or greater than a length of the shaft. The drug eluting apparatus also includes a balloon attached to a distal portion of the shaft and configured to receive the drug from the drug lumen, the balloon including a plurality of holes configured to allow the drug to exit the balloon. The handle includes an actuator that is actuatable by a user to pull the shaft and thereby pull the balloon into the carrier tube and cause the balloon to change from a radially expanded state to a radially compressed state. The method also includes inserting the distal portion of the shaft and the distal portion of the carrier tube into a vessel. The method also includes pushing the shaft or pulling the carrier tube so that the distal portion of the shaft and the balloon are outside of the carrier tube. The method also includes providing the drug to the balloon via the drug lumen so as to inflate the balloon and deliver the drug into the vessel via the plurality of holes of the balloon. The method also includes compressing the balloon by using the actuator to pull the shaft, thereby pulling the balloon into the carrier tube, causing the balloon to change from a radially expanded state to a radially compressed state, and expelling at least a portion of the drug remaining in the balloon.

In some embodiments, a method of delivering a drug into a vessel includes providing a drug eluting apparatus that includes a handle including a drug port configured to receive a drug. The drug eluting apparatus also includes a shaft connected to the handle, the shaft including a drug lumen configured to receive the drug from the drug port, and a guidewire tube configured to receive a guidewire and allow the guidewire to extend out of a distal portion of the shaft. The drug eluting apparatus also includes a carrier tube connected to or connectable to the handle, the carrier tube surrounding the shaft. The carrier tube has a length that is less than a length of the shaft. The drug eluting apparatus also includes a balloon attached to a distal portion of the shaft and configured to receive the drug from the drug lumen, the balloon including a plurality of holes configured to allow the drug to exit the balloon. The handle includes an actuator that is actuatable by a user to pull the shaft and thereby pull the balloon into the carrier tube and cause the balloon to change from a radially expanded state to a radially compressed state. The method also includes inserting the distal portion of the shaft and the distal portion of the carrier tube into a vessel. The method also includes pushing the shaft so that the distal portion of the shaft and the balloon are outside of the carrier tube and the guiding catheter. The method also includes providing the drug to the balloon via the drug lumen so as to inflate the balloon and deliver the drug into the vessel via the plurality of holes of the balloon. The method also includes compressing the balloon by using the actuator to pull the shaft, thereby pulling the balloon into the guiding catheter, causing the balloon to change from a radially expanded state to a radially compressed state, and expelling at least a portion of the drug remaining in the balloon.

In some embodiments, a method of delivering a drug into a vessel includes providing a drug eluting apparatus that includes a handle including a drug port configured to receive a drug. The drug eluting apparatus also includes a shaft connected to the handle. The shaft includes a drug lumen configured to receive the drug from the drug port, and a guidewire tube configured to receive a guidewire and allow the guidewire to extend out of a distal portion of the shaft. The drug eluting apparatus also includes a balloon attached to the distal portion of the shaft and configured to receive the drug from the drug lumen, the balloon including a plurality of holes configured to allow the drug to exit the balloon. The drug eluting apparatus also includes an anchor connected to a distal portion of the balloon. The drug eluting apparatus also includes a pull wire connected to the anchor. The handle includes an actuator that is actuatable by a user to pull the pull wire and thereby cause the balloon to change from a longitudinally expanded state to a longitudinally compressed state. The longitudinally compressed state of the balloon is an inverted state. The method also includes inserting the distal portion of the shaft into a vessel. The method also includes providing the drug to the balloon via the drug lumen so as to inflate the balloon and deliver the drug into the vessel via the plurality of holes of the balloon. The method also includes compressing the balloon by using the actuator to pull the pull wire, thereby causing the balloon to change from the longitudinally expanded state to the longitudinally compressed state, and expelling at least a portion of the drug remaining in the balloon. The longitudinally compressed state of the balloon is an inverted state.

In some embodiments, a method of delivering a drug into a vessel includes providing a drug eluting apparatus that includes a handle including a drug port configured to receive a drug. The drug eluting apparatus also includes a first shaft connected to the handle, the first shaft including a drug lumen configured to receive the drug from the drug port. The drug eluting apparatus also includes a second shaft connected to the handle, the second shaft configured to extend through and move independently of the first shaft, and the second shaft configured to receive a guidewire and allow the guidewire to extend out of a distal portion of the second shaft. The drug eluting apparatus also includes a balloon including a proximal portion connected to a distal portion of the first shaft, and a distal portion connected to a distal portion of the second shaft. The balloon is configured to receive the drug from the drug lumen, and the balloon includes a plurality of holes configured to allow the drug to exit the balloon. The actuator is actuatable by a user to rotate the second shaft relative to the first shaft and thereby cause the balloon to change from a radially expanded state to a radially compressed state. The method also includes inserting the distal portion of the first shaft and the distal portion of the second shaft into a vessel. The method also includes providing the drug to the balloon via the drug lumen so as to inflate the balloon and deliver the drug into the vessel via the plurality of holes of the balloon. The method also includes compressing the balloon by using the actuator to rotate the second shaft relative to the first shaft, thereby causing the balloon to change from the radially expanded state to the radially compressed state and expelling at least a portion of the drug remaining in the balloon.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several implementations in accordance with the disclosure and are therefore, not to be considered limiting of its scope, the disclosure will be described with additional specificity and detail through use of the accompanying drawings.

FIG. 1A is a left side view of a drug eluting apparatus according to a first embodiment in a first configuration, with a balloon expanded to a longitudinally expanded state; FIG. 1B is a left side view of the drug eluting apparatus according to the first embodiment in a second configuration, with the balloon compressed by an anchor into a longitudinally compressed state; and FIG. 1C is a left side view of the drug eluting apparatus according to the first embodiment in a third configuration, after the balloon has returned to the longitudinally expanded state.

FIG. 2A is a front, top, left side perspective view of a handle that may be included in the drug eluting apparatus of the first embodiment; FIG. 2B is a front, top, left side perspective view of the handle of FIG. 2A in another configuration in which the actuator has been adjusted to move pull wires; and FIG. 2C is a front, bottom, left side perspective view of a hub that is included in the handle of FIG. 2A, showing a stopcock.

FIG. 3 is a left side view of a portion of the drug eluting apparatus according to the first embodiment.

FIG. 4A is a cross-sectional view of the portion of the drug eluting apparatus according to the first embodiment taken along the plane A-A in FIG. 3; FIG. 4B is a cross-sectional view of the portion of the drug eluting apparatus according to the first embodiment taken along the plane B-B in FIG. 3; and FIG. 4C is a cross-sectional view of the portion of the drug eluting apparatus according to the first embodiment taken along the plane C-C in FIG. 3.

FIG. 5A is a cross-sectional view of the portion of the drug eluting apparatus according to the first embodiment taken along the plane A-A in FIG. 3; FIG. 5B is a cross-sectional view of the portion of the drug eluting apparatus according to the first embodiment taken along the plane B-B in FIG. 3; and FIG. 5C is a cross-sectional view of the portion of the drug eluting apparatus according to the first embodiment taken along the plane C-C in FIG. 3.

FIG. 6A is a front, top, left side perspective view of a distal portion of the drug eluting apparatus of the first embodiment, with a balloon expanded to a longitudinally expanded state; and FIG. 6B is a cross-sectional front, top, left side perspective view of the distal portion of the drug eluting apparatus of the first embodiment, with the balloon expanded to the longitudinally expanded state, taken along the plane A-A in FIG. 6A.

FIG. 7A is a cross-sectional front view of a distal portion of the drug eluting apparatus of the first embodiment, with the anchor unconnected to the balloon and a guidewire tube, taken along the plane A-A in FIG. 6B. FIG. 7B is a cross-sectional front view of a distal portion of the drug eluting apparatus of the first embodiment, with the anchor unconnected to the balloon and the guidewire tube, taken along the plane A-A in FIG. 6C. FIG. 7C is a cross-sectional front view of the distal portion of the drug eluting apparatus of the first embodiment, with the anchor connected to the balloon and the guidewire tube, taken along the plane A-A in FIG. 6B or 6C.

FIG. 8A is a left side view of a drug eluting apparatus according to a second embodiment in a first configuration, with a shaft in a carrier tube; FIG. 8B is a left side view of the drug eluting apparatus according to the second embodiment in a second configuration, with a distal end of the shaft positioned distal to the carrier tube; FIG. 8C is a left side view of the drug eluting apparatus according to the second embodiment in a third configuration, with a balloon expanded to a radially expanded state; and FIG. 8D is a left side view of a drug eluting apparatus according to the second embodiment in a fourth configuration, with the balloon being pulled into the carrier tube into a radially compressed state.

FIG. 9A is a front, top, left side perspective view of a handle that may be included in the drug eluting apparatus of the second embodiment; and FIG. 9B is a front, bottom, left side perspective view of a hub that is included in the handle of FIG. 9A, showing a stopcock.

FIG. 10A is a right side view of a portion of the drug eluting apparatus according to the second embodiment in a first configuration, with a balloon expanded to a radially expanded state; FIG. 10B is a right side view of a portion of the drug eluting apparatus according to the second embodiment in a second configuration, with the balloon being radially compressed against a carrier tube and partially withdrawn into the carrier tube; and FIG. 10C is a right side view of a portion of the drug eluting apparatus according to the second embodiment in a third configuration, with the balloon radially compressed against the carrier tube and more fully withdrawn into the carrier tube.

FIG. 11A is a left side view of a drug eluting apparatus according to a variation of the second embodiment in a first configuration, with a balloon expanded to a radially expanded state; and FIG. 11B is a left side view of a drug eluting apparatus according to the variation of the second embodiment in a second configuration, with the balloon being pulled in a carrier tube into a radially compressed state.

FIG. 12 is a front, top, left side perspective view of a distal portion of a drug eluting apparatus according to the variation of the second embodiment, with a balloon expanded to a radially expanded state.

FIG. 13A is a left side view of a drug eluting apparatus according to a third embodiment; FIG. 13B is a left side view of a portion of the drug eluting apparatus according to the third embodiment in a first configuration, with a balloon expanded to a longitudinally expanded state; and FIG. 13C is a left side view of a portion of the drug eluting apparatus according to the third embodiment in a second configuration, with the balloon longitudinally compressed into an inverted state.

FIG. 14A is a front, top, left side perspective view of a distal portion of the drug eluting apparatus of the third embodiment, with a balloon expanded to a longitudinally expanded state; and FIG. 14B is a cross-sectional left side view of the distal portion of the drug eluting apparatus of the third embodiment, with the balloon expanded to the longitudinally expanded state, taken along the plane A-A in FIG. 14A.

FIG. 15A is a left side view of a portion of a drug eluting apparatus according to a fourth embodiment in a first configuration, with a balloon expanded to a radially expanded state; and FIG. 15B is a left side view of the portion of the drug eluting apparatus according to the fourth embodiment in a second configuration, with the balloon compressed into a radially compressed state.

FIG. 16 is a left side view of a cross-section of a handle that may be included in the drug eluting apparatus of the fourth embodiment.

FIG. 17A is a right side view of a drug eluting apparatus for use with a short carrier tube and a guiding catheter, in a first configuration; FIG. 17B is a right side view of the drug eluting apparatus for use with the short carrier tube and the guiding catheter, in a second configuration; FIG. 17C is a right side view of the drug eluting apparatus for use with the short carrier tube and the guiding catheter, in a third configuration; and FIG. 17D is a right side view of the drug eluting apparatus for use with the short carrier tube and the guiding catheter, in a fourth configuration.

FIG. 18A is a right side view of a drug eluting apparatus for use with a long carrier tube, in a first configuration; FIG. 18B is a right side view of the drug eluting apparatus for use with the long carrier tube, in a second configuration; FIG. 18C is a right side view of the drug eluting apparatus for use with the long carrier tube, in a third configuration.

FIG. 19A is a right side view of a distal portion of a drug eluting apparatus for use with an inserter, a valve, a guiding catheter, and a flushing device in a first configuration;

FIG. 19B is a right side view of the distal portion of the drug eluting apparatus for use with the inserter, the valve, the guiding catheter, and the flushing device, in a second configuration; FIG. 19C is a right side view of the distal portion of the drug eluting apparatus for use with the inserter, the valve, the guiding catheter, and the flushing device, in a third configuration; and FIG. 19D is a right side view of the distal portion of the drug eluting apparatus for use with the inserter, the valve, the guiding catheter, and the flushing device, in a fourth configuration.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

To reduce costs associated with drug eluting apparatuses, it may be desirable to reduce the amount of a drug that remains in a balloon after the balloon is expanded in a vessel. However, it can be difficult to deliver the drug into the vessel without leaving a portion of the drug remaining in the balloon. Any drug that remains in the balloon may not be recovered and is not used for another patient, and so any remaining drug in the balloon is wasted.

Various embodiments of the drug eluting apparatuses described herein may provide one or more advantages, including, for example: (1) reducing the expenses associated with operating a drug eluting apparatus by allowing for a reduced amount of a drug to be inserted into the drug eluting apparatus; and (2) facilitating the ability of a user of a drug eluting apparatus to operate a drug eluting apparatus.

Various embodiments of the drug eluting apparatuses described herein may be used with a guiding catheter. A guiding catheter may be inserted into a vessel before the drug eluting apparatuses. The drug eluting apparatuses may be connected to the guiding catheter to facilitate entry of portions of the drug eluting apparatuses into the vessel. Use of a guiding catheter may protect the vessel from potential trauma that could be caused by the use of the drug eluting apparatuses. The guiding catheter may be positioned at or near the target of drug elution.

Various embodiments of the drug eluting apparatuses described herein may be used with a carrier tube. A carrier tube may surround a shaft of and/or the balloon of the drug eluting apparatuses. The carrier tube may facilitate entry of portions of the drug eluting apparatuses into the vessel. Use of a carrier tube may provide an external force on the balloon to hold the balloon in a deformed radially compressed state. The carrier tube may be positioned at or near the target of drug elution.

As described herein the term “proximal end” refers to an end that is proximate to a user of a drug eluting apparatus and the term “distal end” refers to an end that is proximate to a subject on which a drug elution procedure is being performed via the drug eluting apparatus.

### First Embodiment

FIG. TA-7C depict a drug eluting apparatus 100 (e.g., treatment apparatus, etc.) for delivering a drug (e.g., a drug that includes an anti-proliferative agent such as sirolimus, etc.) into a vessel according to a first embodiment. The drug eluting apparatus 100 includes a handle 106 having a drug port 104 configured to receive a drug. The drug eluting apparatus 100 further includes a shaft 110 connected to the handle 106. The shaft 110 has a drug lumen 114 configured to receive the drug from the drug port 104, and a guidewire tube 116 configured to receive a guidewire 126 and allow the guidewire 126 to extend out of a distal portion 110d of the shaft 110. The drug eluting apparatus 100 further includes a balloon 130 attached to the distal portion 110d of the shaft 110 and configured to receive the drug from the drug lumen 114, the balloon 130 having a plurality of holes 132 to allow the drug to exit the balloon 130. The drug eluting apparatus 100 further includes an anchor 140 connected to a distal portion of the balloon 130. The drug eluting apparatus 100 further includes an elastically deformable wire 150 having a distal end 150d connected to the distal portion 130d of the balloon 130, and a proximal end 150p connected to the guidewire tube 116. The drug eluting apparatus 100 further includes a pull wire 128 connected to the anchor 140. The handle 106 further includes an actuator 108 that is actuatable by a user to pull the pull wire 128 and thereby cause the balloon 130 to change from a longitudinally expanded state 134a to a longitudinally compressed state 134b. After the pull wire 128 is pulled and the balloon 130 is compressed, the elastically deformable wire 150 causes the balloon 130 to return to the longitudinally expanded state 134a. In some embodiments, the drug eluting apparatus 100 may further include a stopcock 102 connected to the drug port 104. In some embodiments, the shaft 110 may further include a pull wire lumen 118 and the pull wire 128 is located in the pull wire lumen 118 of the shaft 110.

The drug eluting apparatus 100 includes a handle 106 (e.g., grip, handgrip, etc.). The handle 106 is configured to be held by a user and allows a user to operate the drug eluting apparatus 100. The handle 106 may be configured to be held with one hand, so as to allow a user to perform additional maneuvers with the user's second hand.

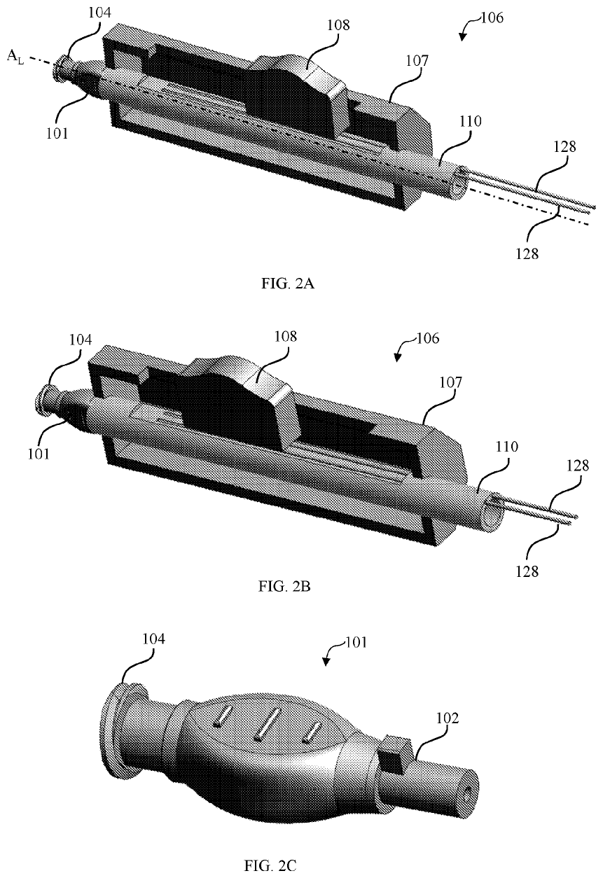

In FIG. 2A-2B, the handle 106 includes a body 107 (e.g. shell, housing, container, etc.). The body 107 of the handle 106 holds together the hub 101, the stopcock 102, the drug port 104, and the actuator 108. The body 107 of the handle 106 allows a user to hold the handle 106 for operation of the drug eluting apparatus 100.

The body 107 may be made of polyethylene, polyolefins (e.g., polypropylene, polyamides), polyesters (e.g., polyethylene terephthalate), fluoropolymers (such as polytetrafluoroethylene (PTFE)), ethylene/tetrafluoroethylene copolymer (ETFE), polyetheretherketone (PEEK), polyamides, elastomer, polyester elastomer, polycarbonate, polyetherimide, polystyrene, polyacetal, ABS (acrylonitrile, butadiene, styrene copolymer synthetic) resin, or other suitable materials.

The handle 106 also includes a hub 101. In FIG. 2A, the hub is located at a proximal side of the handle 106. However, in other embodiments, the hub 101 may be located at the top, bottom, left, right, or proximal side of the handle 106. The hub 101 includes a drug port 104 (e.g., hole, opening, aperture, etc.) configured to receive a drug. The hub 101 may also include a stopcock 102 (e.g., valve, etc.) as in FIG. 2C. The stopcock 102 allows a user of the drug eluting apparatus 100 to control the flow of a drug into the drug eluting apparatus 100. When the stopcock 102 is in a closed position, the drug lumen 114 is sealed so that the drug cannot flow in a proximal or distal direction past the stopcock 102. Therefore, when the balloon 130 is compressed, the drug is forced to flow out of the balloon 130 via the plurality of holes 132, rather than flowing in a proximal direction through the drug lumen 114. The stopcock 102 is located distal of the drug port 104, so that the drug may be provided to the drug port 104 and flow through the stopcock 102 when the stopcock 102 is open. In other embodiments, the stopcock 102 may be provided separate from the hub 101. For example, the hub 101 may be located on a proximal side of the handle 106, and the stopcock 102 is located on a distal side of the handle 106.

The handle 106 also includes an actuator 108 (e.g., tab, button, wheel, knob, etc.). The actuator 108 may be a mechanical structure that is actuatable by a user to operate the drug eluting apparatus 100. The actuator 108 is actuated by a user of the drug eluting apparatus 100 by moving the actuator 108 proximally or distally along the length of the handle 106 in a direction parallel to the longitudinal axis A_{L}. In other embodiments, the actuator 108 may be a wheel that is actuated by a user of the drug eluting apparatus 100 by rolling the wheel in a direction parallel to the longitudinal axis A_{L}. In FIG. 2B, the actuator 108 has been moved proximally as compared to in FIG. 2A, resulting in pulling the pull wire 128 proximally to pull the anchor 140 to longitudinally compress the balloon 130 and cause the drug to exit the plurality of holes 132 of the balloon 130. In FIG. 2A, the actuator 108 is located at a top side of the handle 106. However, in other embodiments, the actuator 108 may be located at a bottom, left, or right side of the handle 106.

The drug eluting apparatus 100 also includes a shaft 110 (e.g., catheter, tube, pipe, cylinder, conduit, etc.). The shaft 110 is connected to the handle 106. The shaft 110 may be connected to the handle 106 by bonding with an adhesive, by an interlocking feature, by press fit, by threaded fastener, or by other suitable connecting arrangements. The shaft may be permanently fixed to the handle 106 so that the user may not disconnect the shaft 110 from the handle 106.

The shaft 110 may be made of a fluorine polymer (e.g., polytetrafluoroethylene (PTFE)), ethylene/tetrafluoroethylene copolymer (ETFE), polyetheretherketone (PEEK), polyurethane, polyamide elastomer, polyester elastomer, polyurethane elastomer, polyimide, polycarbonate, polyetherimide, fluorocarbon resin, or other suitable materials.

The shaft 110 may have a length that is approximately in a range of 30 to 300 cm, for example, 50-300 cm, or 100-250 cm. As used herein, a range of X to Y includes X, Y, and values between X and Y.

The shaft 110 may have a cross-sectional shape that is a circle, an oval, a polygon, a rounded polygon, or other geometric shape. The shaft 110 may have a maximum cross-sectional width (e.g., a diameter where the cross-sectional shape is circular) that is approximately in a range of 0.5-10 mm, inclusive, and ideally 0.5-7 mm.

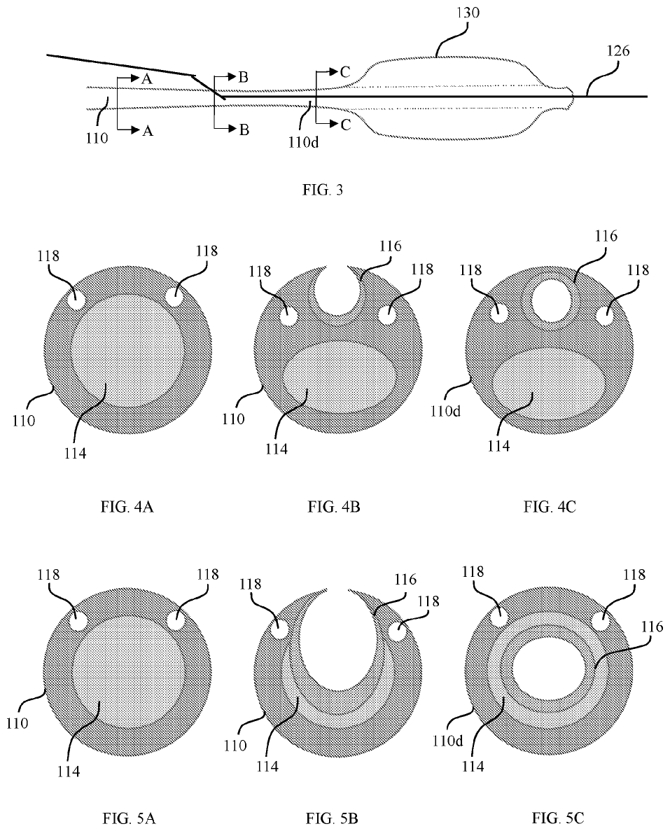

FIG. 3 shows a portion of the drug eluting apparatus 100. The drug eluting apparatus 100 may be a rapid-exchange type device, in which the guidewire 126 enters the shaft 110 at a location of the shaft 110 that is distal to the location where the shaft 110 is connected to the handle 106. In other embodiments, the drug eluting apparatus 100 may be an over-the-wire type device, in which the guidewire 126 enters the shaft 110 through the handle 106. The guidewire tube 116, which is configured to receive the guidewire 126, extends longitudinally through a portion of the length of the shaft 110.

In FIGS. 4A-4C and FIGS. 5A-5C, the shaft 110 includes a drug lumen 114 (e.g., cavity, space, etc.), and a guidewire tube 116 (e.g., pipe, cylinder, conduit, etc.). In some embodiments, as in FIGS. 4A-4C and FIGS. 5A-5C, the shaft 110 includes a pull wire lumen 118 (e.g., cavity, space, etc.), FIGS. 4A-4C show cross-sections of the shaft 110 at planes A-A, B-B, and C-C respectively of FIG. 3. In the embodiment of FIGS. 4A-4C, the guidewire tube 116 is separate from the drug lumen 114. In the embodiment of FIGS. 5A-5C, the guidewire tube 116 extends through the drug lumen 114. FIGS. 5A-5C show cross-sections of another shaft 110 at planes A-A, B-B, and C-C respectively of FIG. 3.

The drug lumen 114 extends longitudinally through the shaft 110. The drug lumen 114 is configured to receive the drug from the drug port 104. The drug lumen 114 may have a cross-sectional shape that is a circle, an oval, a polygon, a rounded polygon, or other geometric shape. The drug lumen 114 may have a maximum cross-sectional width (e.g., a diameter where the cross-sectional shape is circular) that is approximately in a range of 1 μm to 15 mm, inclusive, and ideally 0.1-3 mm.

In some embodiments, the shaft 110 has the pull wire lumen 118 that extends longitudinally through the shaft 110. The pull wire lumen 118 may have a cross-sectional shape that is a circle, an oval, a polygon, a rounded polygon, or other geometric shape. The pull wire lumen 118 may have a maximum cross-sectional width (e.g., a diameter where the cross-sectional shape is circular) that is approximately in a range of 0.1-10 mm, inclusive, and ideally 0.1-3 mm. In FIGS. 4A-4C and FIGS. 5A-5C, the shaft 110 has two pull wire lumens 118. However, in other embodiments, the shaft 110 may have one, two, three, or more pull wire lumens 118.

The guidewire tube 116 is configured to receive a guidewire 126 and allow the guidewire 126 to extend out of a distal portion 110d of the shaft 110, as in FIG. 3. The guidewire tube 116 extends longitudinally through a portion of the shaft 110. The guidewire tube 116 may have a cross-sectional shape that is a circle, an oval, a polygon, a rounded polygon, or other geometric shape. The guidewire tube 116 may have a maximum cross-sectional width (e.g., a diameter where the cross-sectional shape is circular) that is approximately in a range of 0.1-1 mm, inclusive, and ideally 0.35-0.9 mm.

In FIGS. 4A-4C, the drug lumen 114, the guidewire tube 116, and the pull wire lumens 118 are positioned in the shaft 110 such that the drug lumen 114 is in the center of the shaft 110, the guidewire tube 116 is at the top of the shaft 110, and the pull wire lumens 118 are at the top of the shaft 110. In other embodiments, the drug lumen 114, the guidewire tube 116, and the pull wire lumen 118 may be positioned at other locations of the shaft 110 and with different relative positioning to each other, as compared to the arrangement of FIGS. 4A-4C. In FIGS. 5A-5C, the drug lumen 114, the guidewire tube 116, and the pull wire lumens 118 are positioned in the shaft 110 such that the drug lumen 114 is in the center of the shaft 110, the guidewire tube 116 is at the top and center of the shaft 110, and the pull wire lumens 118 are at the top of the shaft 110. In other embodiments, the drug lumen 114, the guidewire tube 116, and the pull wire lumen 118 may be positioned at other locations of the shaft 110 and with different relative positioning to each other, as compared to the arrangement of FIGS. 5A-5C.

The drug eluting apparatus 100 also includes a balloon 130. The balloon 130 is attached to the distal portion 110d of the shaft 110. The balloon 130 may be attached to the distal portion 110d of the shaft 110 by being placed over the distal portion 110d of the shaft 110 and bonded with an adhesive, by welding, or by other suitable connecting arrangements. The balloon 130 is configured to receive the drug from the drug lumen 114. When the drug enters the balloon 130 from the drug lumen 114, the balloon 130 expands to a longitudinally expanded state 134a. The balloon 130 may have a preset fold or a crease to guide the shape of the balloon 130 as the balloon 130 is longitudinally compressed. The preset folds or creases of the balloon 130 may be configured to allow the balloon 130 to be longitudinally compressed in an ordered way.

The balloon 130 may be made of polyamide, polyester, polyurethane, polyamide elastomer, polyester elastomer, polyurethane elastomer, or other suitable materials.

The balloon 130 may have a length that is approximately in a range of 10-400 mm, for example, 10-50 mm, or 15-40 mm. The balloon 130 may have a width that is approximately in a range of 1-8 mm, for example, 2-5 mm, or 4-8 mm.

The balloon 130 includes a plurality of holes 132 (e.g., apertures, openings, slits, etc.) that are configured to allow the drug to exit the balloon 130. When the balloon 130 is expanded to the longitudinally expanded state 134a, the drug passes through the plurality of holes 132 of the balloon 130 to exit the balloon 130 and enter the vessel.

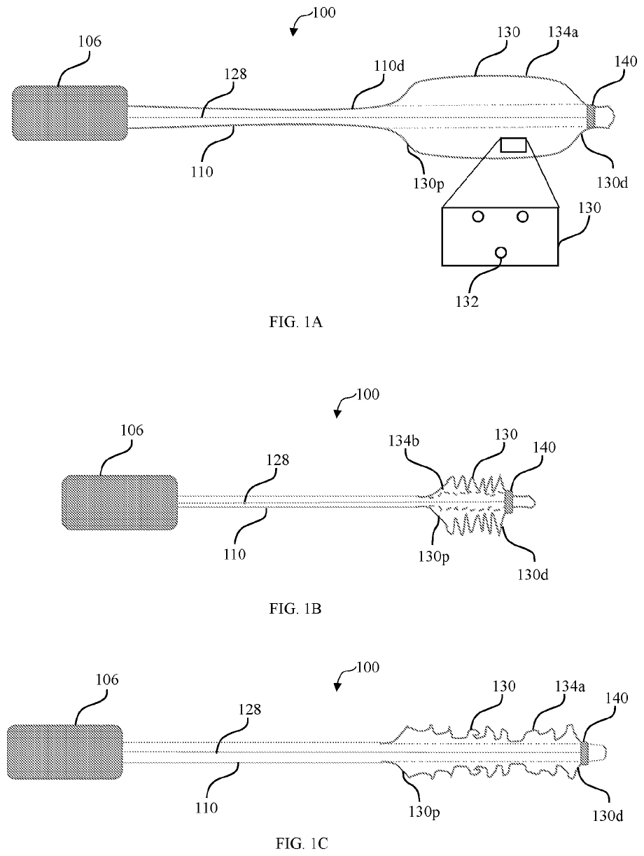

The plurality of holes 132 of the balloon 130 may each have a width that is approximately in a range of 100 nm to 10 μm, and ideally 100 nm to 5 μm. In FIG. 1A, the plurality of holes 132 of the balloon 130 have the same widths. However, in other embodiments, the plurality of holes 132 of the balloon 130 may have different widths.

The plurality of holes 132 of the balloon 130 may each have a shape that is a circle, an oval, a rounded polygon, a slit, or other geometric shape. In FIG. 1A, the plurality of holes 132 of the balloon 130 have the same shape. However, in other embodiments, the plurality of holes 132 of the balloon 130 may have different shapes.

In FIG. 1A, the plurality of holes 132 of the balloon 130 are positioned with a uniform spacing between the plurality of holes 132 of the balloon 130. However, in other embodiments, the plurality of holes 132 of the balloon 130 may be positioned with a different spacing between the plurality of holes 132 of the balloon 130.

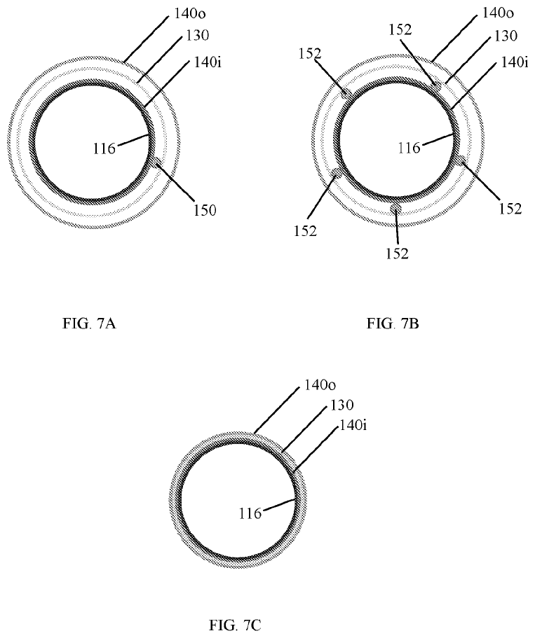

The drug eluting apparatus 100 also includes an anchor 140 (e.g., block, weight, cap, etc.). The anchor 140 is connected to a distal portion 130d of the balloon 130. The anchor 140 may be connected to the distal portion 130d of the balloon 130 by swaging, by crimping, or by other suitable connecting arrangements. In FIG. 7A-7B, the anchor 140 includes an inner portion 140i and an outer portion 140o. The balloon 130 and the elastically deformable wire 150 may be positioned between the inner portion 140i of the anchor 140 and the outer portion 140o of the anchor 140. The inner portion 140i of the anchor 140 may be positioned surrounding the guidewire tube 116. The guidewire tube 116, the inner portion 140i of the anchor 140, the elastically deformable wire 150 or the plurality of elastically deformable wire portions 152, the balloon 130, and the outer portion of the 140o of FIG. 7A or 7B may be connected by swaging, by crimping, or by other suitable connecting arrangements so that these components are connected as in the configuration of FIG. 7C. In FIG. 7C, the elastically deformable wire 150 or the plurality of elastically deformable wire portions 152 are in between the inner portion 140i of the anchor 140 and the balloon 130, which are swaged. In some embodiments, the balloon 130 may include a tip shaft where the balloon 130 connects to the anchor 140, with the tip shaft allowing the distal portion of the drug eluting apparatus 100 to be less likely to cause trauma to the vessel.

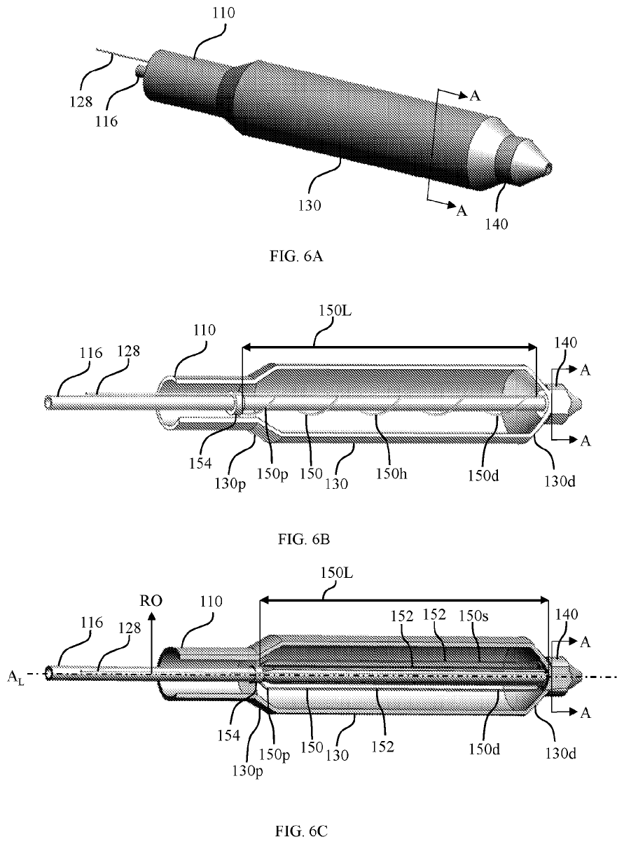

The anchor 140 may be made of plastic, nitinol, gold, platinum, stainless steel, tungsten, tantalum, or other suitable materials. The anchor 140 may have a width that is approximately in a range of 0.5-10 mm, for example, 1-4 mm. In FIG. 6A, the anchor 140 has a shape that is a cylinder. However, in other embodiments, the anchor 140 may have a shape that is a cylinder, an elliptic cylinder, a hemisphere, or other shape.

The drug eluting apparatus 100 also includes an elastically deformable wire 150 (e.g., thread, strand, line, filament, etc.). The elastically deformable wire 150 has a distal end 150d connected to the anchor 140, and the anchor 140 is connected to the distal portion 130d of the balloon 130, as in FIG. 7A-7C. A proximal end 150p of the elastically deformable wire 150 may be connected to the guidewire tube 116 via a marker band 154 (e.g., hoop, band, disk, etc.) and may be connected via blading, swaging, press-fit, welding, crimping, or other suitable connecting arrangements.

The marker band 154 may be made of gold, platinum, stainless steel, tungsten, tantalum, or other suitable materials. The marker band 154 may be made of a radiopaque material. When the marker band 154 is made of a radiopaque material, the marker band 154 may be imaged using an imaging modality such as X-ray, fluoroscopy, or other suitable imaging modality. The radiopaque material of the marker band 154 facilitates the detection and tracking via imaging of the distal portion 110d of the shaft 110 when the distal portion 110d of the shaft 110 is inside a vessel. Such detection and tracking of the marker band 154 may allow for improved operation and positioning of the drug eluting apparatus 100 within the vessel and for improved targeting of the location of a portion of a vessel, as a user may have information of where the distal portion 110d of the shaft 110 is located through knowledge of the location of the marker band 154 via imaging.

The elastically deformable wire 150 may be made of nitinol or stainless steel. The elastically deformable wire 150 may have a width that is approximately in a range of 0.1 mm-2 mm, for example, 0.5-1 mm.

The elastically deformable wire 150 may be in a relaxed longitudinally expanded state, or in a deformed longitudinally collapsed state. The deformed state is the state in which an external force is applied to the elastically deformable wire 150. The relaxed state is the state in which no external force is applied to the elastically deformable wire 150. When in a relaxed state, as when the elastically deformable wire 150 is not being compressed, the elastically deformable wire 150 is in a relaxed longitudinally expanded state to support the longitudinally expanded shape of the balloon 130 in the longitudinally expanded state 134a. In FIG. 6B, where the balloon 130 is in the longitudinally expanded state 134a, the elastically deformable wire 150 is in a relaxed longitudinally expanded state. When in a deformed state, as when the elastically deformable wire 150 is being compressed by the anchor 140 and so restricted in shape in the longitudinally collapsed state, the elastically deformable wire 150 is in a deformed longitudinally collapsed state.

The elastically deformable wire 150 may have a spring constant that varies along a length 150L of the elastically deformable wire 150. When the spring constant varies along the length 150L of the elastically deformable wire 150, when the elastically deformable wire 150 is longitudinally compressed, the portion of the elastically deformable wire 150 having the lower spring constant is longitudinally compressed before the portion of the elastically deformable wire 150 having the higher spring constant.

In some embodiments, the elastically deformable wire 150 may have a higher spring constant at a proximal end 150p of the elastically deformable wire 150 than at a distal end 150d of the elastically deformable wire 150. When the spring constant of the proximal end 150p of the elastically deformable wire 150 is higher than the spring constant of the distal end 150d of the elastically deformable wire 150, when the wire is longitudinally compressed, the distal end 150d of the elastically deformable wire 150 is longitudinally compressed before the proximal end 150p of the elastically deformable wire 150 is longitudinally compressed. Longitudinally compressing the distal end 150d of the elastically deformable wire 150 before longitudinally compressing the proximal end 150p of the elastically deformable wire 150 facilitates preventing hindering or blocking of the drug pathway through the balloon 130 that could result in reduced flow of the drug through the drug eluting apparatus 100. In other embodiments, the elastically deformable wire 150 may have a lower spring constant at a proximal end 150p of the elastically deformable wire 150 than at a distal end 150d of the elastically deformable wire 150. In still other embodiments, the elastically deformable wire 150 may have a different spring constant at a plurality of points along the length 150L of the elastically deformable wire 150.

The distal end 150d of the elastically deformable wire 150 may be attached to the distal portion 130d of the balloon 130 via the anchor 140. The distal end 150d of the elastically deformable wire 150 may be attached to the anchor 140 by swaging, or by other suitable connecting arrangements.

The elastically deformable wire 150 is in the balloon 130 as in FIG. 6B-6C. Before the balloon 130 is inflated to the longitudinally expanded state 134a, the elastically deformable wire 150 may be in a deformed longitudinally collapsed state in the balloon 130. In the deformed state, the elastically deformable wire 150 is in a deformed longitudinally collapsed state. After the balloon 130 is inflated to the longitudinally expanded state 134a, the elastically deformable wire 150 is in a relaxed longitudinally expanded stated. In the relaxed state, the elastically deformable wire 150 expands to a relaxed longitudinally expanded state to support the longitudinally expanded state 134a of the balloon 130.

In FIG. 6B, the elastically deformable wire 150 has a helical shape 150h. In a helical shape 150h, the elastically deformable wire 150 has a shape that consists of a series of rotations from the proximal end 150p of the elastically deformable wire 150 to the distal end 150d of the elastically deformable wire 150. The rotations of the helical shape 150h may be circular, elliptical, or other rounded arrangements.

In FIG. 6C, the elastically deformable wire 150 has a straight shape 150s. In a straight shape 150s, the elastically deformable wire 150 includes a plurality of elastically deformable wire portions 152. The plurality of elastically deformable wire portions 152 extend from a proximal portion 130p of the balloon 130 to a distal portion 130d of the balloon 130 and are curved radially outward in a direction RO relative to a longitudinal axis A_{L} of the shaft 110. The plurality of elastically deformable wire portions 152 may have widths that are approximately in a range of 0.1-2 mm, for example, 0.5-1 mm. In FIG. 6C, the plurality of elastically deformable wire portions 152 are each shown as having an approximately similar width. However, in other embodiments, the plurality of elastically deformable wire portions 152 may have varying widths. In FIG. 6C, the plurality of elastically deformable wire portions 152 have six elastically deformable wire portions 152. In other embodiments, the plurality of elastically deformable wire portions 152 may have one, two, three, four, five, six, seven, eight, nine, or more elastically deformable wire portions 152. When the elastically deformable wire 150 having the straight shape 150s is longitudinally compressed, the plurality of elastically deformable wire portions 152 deform radially outward in a direction RO relative to a longitudinal axis A_{L} of the shaft 110.

The drug eluting apparatus 100 also includes a pull wire 128 (e.g., thread, strand, line, filament, etc.). In FIG. 2A, the drug eluting apparatus 100 includes two pull wires 128. In other embodiments, the drug eluting apparatus 100 may include one, two, three, or more pull wires. In some embodiments, the pull wire 128 is located in the drug lumen 114 of the shaft 110. In other embodiments, the pull wire 128 is located in the pull wire lumen 118 of the shaft 110. In some embodiments, a single pull wire 128 may be located in the drug lumen 114 of the shaft 110. In other embodiments, multiple pull wires 128 may be located in the drug lumen 114 of the shaft 110. In some embodiments, a single pull wire 128 may be located in a single pull wire lumen 118 of the shaft 110. In other embodiments, multiple pull wires 128 may be located in a single pull wire lumen 118 of the shaft 110. The pull wire 128 is connected to the anchor 140. Connecting the pull wire 128 to the anchor 140 allows for the anchor 140 to move proximally and to compress the balloon 130 when the pull wire 128 is pulled.

The pull wire 128 may be pulled by the actuator 108 when a user actuates the actuator 108. Pulling the pull wire 128 thereby causes the balloon 130 to change from a longitudinally expanded state 134a to a longitudinally compressed state 134b. In FIG. 1A, the balloon 130 is in a longitudinally expanded state 134a. In FIG. 1B, the balloon 130 is in a longitudinally compressed state 134b. As the balloon 130 changes from the longitudinally expanded state 134a to the longitudinally compressed state 134b, at least a portion of the drug that was remaining in the balloon 130, but that had not yet exited the balloon 130, exits through the plurality of holes 132 of the balloon 130 and enters the vessel.

In FIG. 1A-IC, after the pull wire 128 is pulled and the balloon 130 is compressed to the longitudinally compressed state 134b, the elastically deformable wire 150 causes the balloon 130 to return to the longitudinally expanded state 134a. In FIG. 1A, while and/or after the balloon 130 is inflated with the drug to the longitudinally expanded state 134a, the elastically deformable wire 150 expands to support the longitudinally expanded state 134a of the balloon 130. After the balloon 130 is compressed to the longitudinally compressed state 134b by actuating the actuator 108 to pull the pull wire 128 and move the anchor 140 proximally, as in FIG. 1B, the elastically deformable wire 150 is in a deformed longitudinally collapsed state in the balloon 130 as the elastically deformable wire 150 is also compressed by the anchor 140. The actuator 108 may then cease being actuated such that the pull wire 128 ceases pulling the pull wire 128, allowing the balloon 130 to recover its length as it returns to the longitudinally expanded state 134a, and the elastically deformable wire 150 may expand to support the longitudinally expanded state 134a of the balloon 130, as in FIG. 1C. In its relaxed state, as when the elastically deformable wire 150 is not being compressed by the anchor 140 pulled by the pull wire 128, the elastically deformable wire 150 is in a relaxed longitudinally expanded state.

In some embodiments, the drug eluting apparatus 100 may be used with a long carrier tube 180 (discussed in further detail herein). In other embodiments, the drug eluting apparatus 100 may be used with a short carrier tube 182 (discussed in further detail herein). In some embodiments, the drug eluting apparatus 100 may be used with the long carrier tube 180 or the short carrier tube 182, which is configured to be attached to a guiding catheter 184 (discussed in further detail herein).

### Second Embodiment

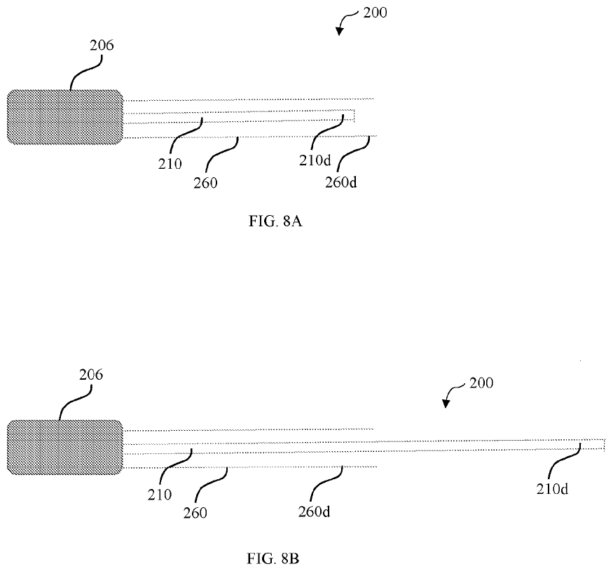

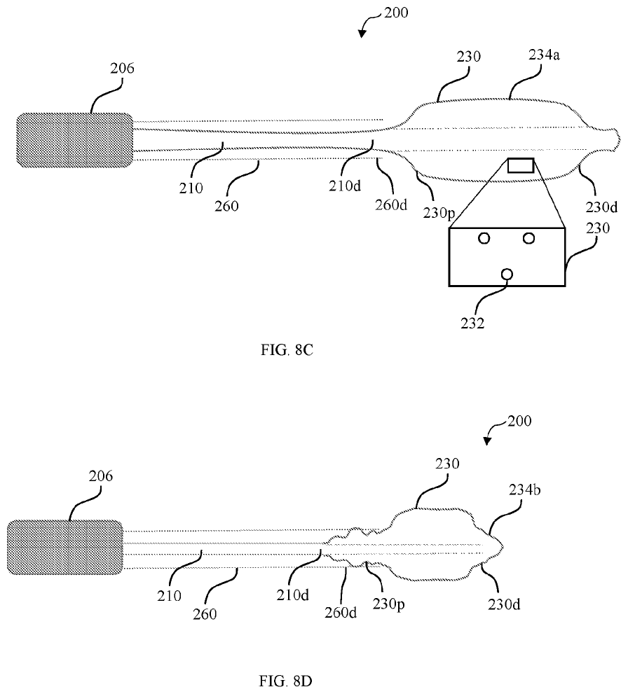

FIGS. 8A-12 depict a drug eluting apparatus 200 (e.g., treatment apparatus, etc.) for delivering a drug (e.g., a drug that includes an anti-proliferative agent such as sirolimus, etc.) into a vessel according to a second embodiment. The drug eluting apparatus 200 includes a handle 206 having a drug port 204 configured to receive a drug. The drug eluting apparatus 200 further includes a shaft 210 connected to the handle 206. The shaft 210 has a drug lumen 214 configured to receive the drug from the drug port 204, and a guidewire tube 216 configured to receive a guidewire 226 and allow the guidewire 226 to extend out of a distal portion 210d of the shaft 210. The drug eluting apparatus 200 further includes a carrier tube 260 connected to or connectable to the handle 206, the carrier tube 260 surrounding the shaft 210. The drug eluting apparatus 200 further includes a balloon 230 attached to a distal portion 210d of the shaft 210 and configured to receive the drug from the drug lumen 214, the balloon 230 including a plurality of holes 232 configured to allow the drug to exit the balloon 230. The handle 206 comprises an actuator 208 that is actuatable by a user to pull the shaft 210 and thereby pull the balloon 230 into the carrier tube 260 and cause the balloon 230 to change from a radially expanded state 234a to a radially compressed state 234b. In some embodiments, the drug eluting apparatus 200 may further include a stopcock 202 connected to the drug port 204. In some embodiments, the drug eluting apparatus 200 may further include one or more elastically deformable wires 256 that extend from a proximal portion 230p of the balloon 230 to a distal portion 230d of the balloon 230 and are curved radially outward relative to a longitudinal axis A_{L} of the shaft 210 when in a relaxed state. In some embodiments, the drug eluting apparatus 200 may further include a ring 270 connected to a distal portion 210d of the carrier tube 260. An elastic modulus of a material of the ring 270 is greater than an elastic modulus of a material of the carrier tube 260.

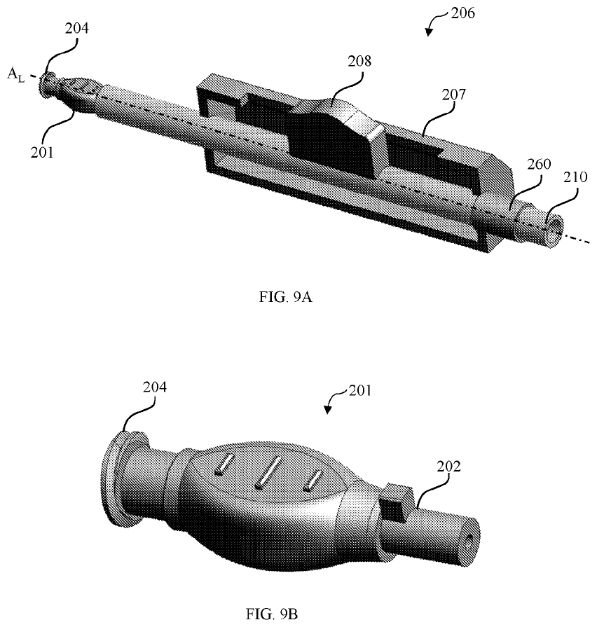

The drug eluting apparatus 200 includes a handle 206 (e.g., grip, handgrip, etc.). The handle 206 is configured in the same way as the handle 106. The handle 206 includes a body 207 (e.g. shell, housing, container, etc.) that is configured in the same way as the body 107. In FIG. 9B, the handle 206 also includes a hub 201 that is configured in the same way as the hub 101. The hub 201 includes a drug port 204 (e.g., hole, opening, aperture, etc.) that is configured in the same way as the drug port 104. The handle 206 may also include a stopcock 202 (e.g., valve, etc.) that is configured in the same way as the stopcock 102.

The handle 206 also includes an actuator 208 (e.g., tab, button, wheel, knob, etc.). The actuator 208 may be a mechanical structure that is actuatable by a user to operate the drug eluting apparatus 200. The actuator 208 is actuated by a user of the drug eluting apparatus 200 by rotating the actuator 208 in either a clockwise or a counterclockwise direction about the length of the handle 206 in a direction parallel to the longitudinal axis A_{L}. Actuating the actuator 208 results in pulling the shaft 210 proximally to pull the balloon 230 into the carrier tube 260 to radially compress the balloon 230 and cause the drug to exit the plurality of holes 232 of the balloon 230. In FIG. 9A, the actuator 208 is located at a top side of the handle 206. However, in other embodiments, the actuator 208 may be located at a bottom, left, or right side of the handle 206.

The drug eluting apparatus 200 also includes a shaft 210 (e.g., catheter, tube, pipe, cylinder, conduit, etc.). The shaft 210 is connected to the handle 206. The shaft 210 may be connected to the handle 206 by bonding with an adhesive, by an interlocking feature, by press fit, by threaded fastener, or by other suitable connecting arrangements. The shaft 210 may be permanently fixed to the handle 206 so that the user may not disconnect the shaft 210 from the handle 206.

The shaft 210 may be made of a fluorine polymer (e.g., polytetrafluoroethylene (PTFE)), ethylene/tetrafluoroethylene copolymer (ETFE), polyetheretherketone (PEEK), polyurethane, polyamide elastomer, polyester elastomer, polyurethane elastomer, polyimide, polycarbonate, polyetherimide, fluorocarbon resin, or other suitable materials.

The shaft 210 has a length that is approximately in a range of 30 to 300 cm, for example, 30-200 cm.

The shaft 210 may have a cross-sectional shape that is a circle, an oval, a polygon, a rounded polygon, or other geometric shape. The shaft 210 may have a maximum cross-sectional width (e.g., a diameter where the cross-sectional shape is circular) that is approximately in a range of 0.5-10 mm, inclusive, and ideally 0.5-7 mm.

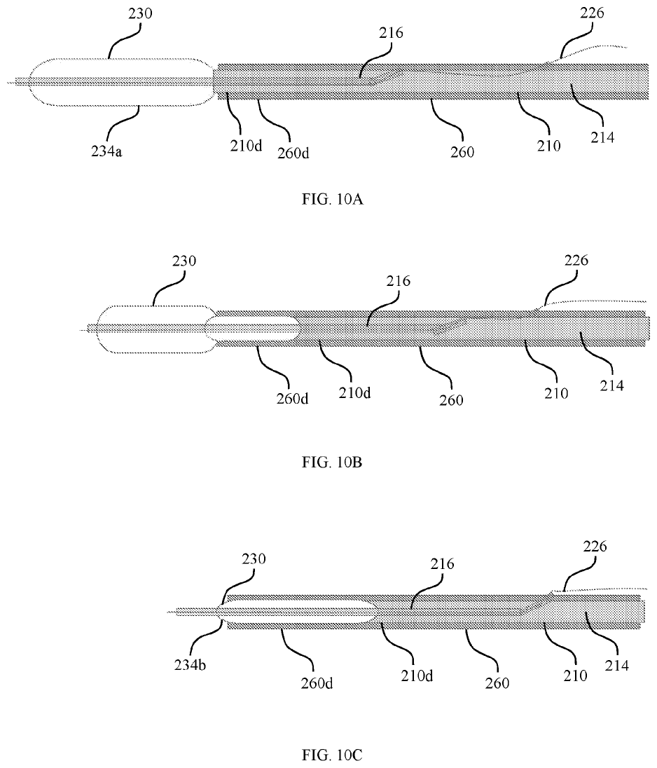

FIGS. 10A-10C show a right side view of a portion of the drug eluting apparatus 200. The drug eluting apparatus 200 may be a rapid-exchange type device, in which the guidewire 226 enters the shaft 210 at a location of the shaft 210 that is distal to the location where the shaft 210 is connected to the handle 206. In other embodiments, the drug eluting apparatus 200 may be an over-the-wire type device, in which the guidewire 226 enters the shaft 210 through the handle 206. The guidewire tube 216, which is configured to receive the guidewire 226, extends longitudinally through a portion of the length of the shaft 210.

The shaft 210 includes a drug lumen 214 (e.g., cavity, space, etc.) and a guidewire tube 216 (e.g., pipe, cylinder, conduit, etc.). The drug lumen 214 is configured in the same way as the drug lumen 114. The guidewire tube 216 is configured in the same way as the guidewire tube 116.

The drug eluting apparatus 200 also includes a carrier tube 260 (e.g., sheath, guiding sheath, cover, etc.). In FIGS. 8A-8D, the carrier tube 260 is connected to the handle 206. In other embodiments, the carrier tube 260 may be unconnected to the handle 206. The carrier tube 260 surrounds the shaft 210 such that the shaft 210 is configured to extend longitudinally through the carrier tube 260. The carrier tube 260 may be connected to the handle 206 by a Luer lock connector, by a lock connector, by a fitting connector, or by other suitable connecting arrangements. The carrier tube 260 may be fixed to the handle 206 so that the user may not disconnect the carrier tube 260 from the handle 206.

The carrier tube 260 may be a long carrier tube 180 (discussed in further detail herein), as in FIGS. 8A-8D. In other embodiments, the carrier tube 260 may be a short carrier tube 182 (discussed in further detail herein). In some embodiments, the carrier tube 260 is configured to be attached to a guiding catheter 184 (discussed in further detail herein).

The carrier tube 260 may be made of a fluorine polymer (e.g., polytetrafluoroethylene (PTFE), ethylene/tetrafluoroethylene copolymer (ETFE), polyetheretherketone (PEEK), polyurethane, polyamide elastomer, polyester elastomer, polyurethane elastomer, polyimide, polycarbonate, polyetherimide, tungsten, stainless steel, Ni—Ti, or other suitable materials.

The carrier tube 260 has a length that is approximately in a range of 30 cm to 300 cm, for example, 30-200 cm.

The carrier tube 260 has a cross-sectional shape that is a circle, an oval, a polygon, a rounded polygon, or other geometric shape. The carrier tube 260 may have a maximum cross-sectional width (e.g., a diameter where the cross-sectional shape is circular) that is approximately in a range of 1-15 mm, inclusive, and for example, 2-8 mm. The carrier tube 260 may have a wall having a thickness that is approximately in a range of 0.1-1 mm, inclusive.

The drug eluting apparatus 200 also includes a balloon 230. The balloon 230 is configured in the same way as the balloon 130. However, when the drug enters the balloon 230 from the drug lumen 214, the balloon 230 expands to a radially expanded state 234a as compared to the longitudinally expanded state 134a of the balloon 130. The balloon 230 includes a plurality of holes 232 (e.g., apertures, openings, slits, etc.) that are configured in the same way as the plurality of holes 132 of the balloon 130.

The actuator 208 that is actuatable by a user to pull the shaft 210 pulls the balloon 230 into the carrier tube 260, thereby causing the balloon 230 to change from a radially expanded state 234a to a radially compressed state 234b. As the balloon 230 is retracted into the carrier tube 260, the balloon is radially compressed against the carrier tube 260. Radially compressing the balloon 230 against the carrier tube 260 compresses the balloon 230, which doing so allows for a smooth compression action and for at least a portion of the drug remaining in the balloon 230 to exit the balloon 230 through the plurality of holes 232 as the balloon 230 changes from the radially expanded state 234a to the radially compressed state 234b.

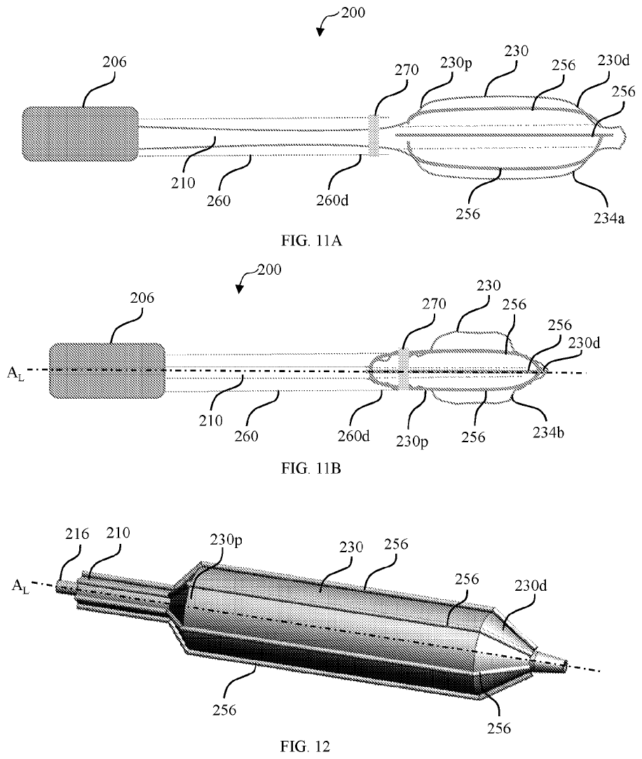

The drug eluting apparatus 200 may further include one or more elastically deformable wires 256 (e.g., thread, strand, line, filament, etc.) that extend from a proximal portion 230p of the balloon 230 to a distal portion 230d of the balloon 230 and are curved radially outward relative to a longitudinal axis A_{L} of the shaft 210 when the one or more elastically deformable wires 256 are in a relaxed state. The deformed state is the state in which an external force is applied to the one or more elastically deformable wires 256, and the relaxed state is the state in which no external force is applied to the one or more elastically deformable wires 256. When in a relaxed state, as when the one or more elastically deformable wires 256 are located outside of the carrier tube 260, the one or more elastically deformable wires 256 are in a relaxed radially expanded state to support the radially expanded state 234a of the balloon 230. When in a deformed state, as when the one or more elastically deformable wires 256 are located inside of the carrier tube 260 and so restricted from expanding by the carrier tube 260, the one or more elastically deformable wires 256 are in a deformed radially collapsed state. While and/or after the balloon 230 is inflated to the radially expanded state 234a, the one or more elastically deformable wires 256 change from a deformed radially collapsed state to a relaxed radially expanded state. In FIGS. 11A-11B, the drug eluting apparatus 200 has three elastically deformable wires 256. In FIG. 12, the drug eluting apparatus 200 has six elastically deformable wires 256. In other embodiments, the drug eluting apparatus 200 may have one, two, three, four, five, six, or more elastically deformable wires 256.

The one or more elastically deformable wires 256 may be made of nitinol or stainless steel. The one or more elastically deformable wires 256 may have widths that are approximately in a range of 0.1-2 mm, inclusive, and for example, 0.3-1 mm. In FIGS. 11A-11B and FIG. 12, the one or more elastically deformable wires 256 have the same widths. However, in other embodiments, the one or more elastically deformable wires 256 may have different widths.

In FIGS. 11A-11B and FIG. 12, the one or more elastically deformable wires 256 are spaced apart at regular intervals around the balloon 230. However, in other embodiments, the one or more elastically deformable wires 256 may be spaced apart at different intervals around the balloon 230.

The drug eluting apparatus 200 may further include a ring 270 (e.g., hoop, band, disk, etc.). In FIGS. 11A-11B, the ring 270 is connected to a distal portion 260d of the carrier tube 260. The ring 270 may be connected to a distal portion 260d of the carrier tube 260 by welding, by blading, by press-fit, by swaging, or by other suitable connecting arrangements. The ring 270 has a maximum cross-sectional width that is approximately in a range of 1-15 mm, inclusive, and for example, 3-8 mm.

The ring 270 has an elastic modulus of a material of the ring 270 that is greater than an elastic modulus of a material of the carrier tube 260. The elastic modulus of a material of the ring 270 being greater than an elastic modulus of a material of the carrier tube 260 allows the ring 270 to provide a more rigid resistance to the balloon 230 as the balloon 230 is pulled into the carrier tube 260, such that the ring 270 provides a greater amount of compression on the balloon 230 and allows for a smoother compression action of the balloon 230, as compared to when the ring 270 is not present.

The ring 270 may be made of gold, platinum, stainless steel, tungsten, tantalum, or other suitable materials. The ring 270 may be made of a radiopaque material. When the ring 270 is made of a radiopaque material, the ring 270 may be imaged using an imaging modality such as X-ray, fluoroscopy, or other suitable imaging modality. The radiopaque material of the ring 270 facilitates the detection and tracking via imaging of the distal portion 260d of the carrier tube 260 when the distal portion 260d of the carrier tube 260 is inside a vessel. Such detection and tracking of the ring 270 may allow for improved operation and positioning of the drug eluting apparatus 200 within the vessel and for improved targeting of the location of a portion of a vessel, as a user may have information of where the distal portion 260d of the carrier tube 260 is located through knowledge of the location of the ring 270 via imaging.

In FIGS. 8A-8D and FIGS. 10A-10C, in one embodiment the drug eluting apparatus 200 includes the carrier tube 260, but does not include the one or more elastically deformable wires 256 or the ring 270. In FIG. 8A, in one configuration the shaft 210 and the carrier tube 260 may be inserted into a vessel such that the distal portion 210d of the shaft 210 is proximal to the distal portion 260d of the carrier tube 260. In FIG. 8B, in another configuration the shaft 210 and the carrier tube 260 may be inserted into a vessel such that the distal portion 210d of the shaft 210 is distal to the distal portion 260d of the carrier tube 260. When the distal portion 210d of the shaft 210 is proximal to the distal portion 260d of the carrier tube 260 as in the configuration of FIG. 8A, the distal portion 210d of the shaft 210 may be moved distally such that the distal portion 210d of the shaft 210 is distal to the distal portion 260d of the carrier tube 260 as in the configuration of FIG. 8B. In FIG. 8C and FIG. 10A, the balloon 230 may be inflated with the drug so that the balloon 230 enters the radially expanded state 234a and the drug exits from the balloon 230 through the plurality of holes 232 of the balloon 230. In FIG. 8D and FIG. 10B-10C, the actuator 208 is actuated to pull the shaft 210 to pull the proximal portion 230p of the balloon 230 into the carrier tube 260 and compress the balloon 230 into the radially compressed state 234b, thereby causing at least a portion of the drug that remained in the radially expanded state 234a of the balloon 230 to exit the balloon 230 through the plurality of holes 232.

In other embodiments, the drug eluting apparatus 200 includes the carrier tube 260 and the one or more elastically deformable wires 256, but does not include the ring 270. The balloon 230 may be inflated with the drug to enter the radially expanded state 234a so that the drug exits through the plurality of holes 232 of the balloon 230 to enter the vessel. As the actuator 208 is actuated to pull the shaft 210 and compress the balloon 230 into the radially compressed state 234b, the balloon 230 is pulled into the carrier tube 260. Pulling the balloon 230 into the carrier tube 260 radially compresses the balloon 230 so that at least a portion of the drug remaining in the balloon 230 exits through the plurality of holes 232 of the balloon. As the actuator 208 is actuated to pull the shaft 210 and radially compress the balloon 230 into the radially compressed state 234b, the one or more elastically deformable wires 256 are compressed, which also radially compresses the balloon 230. The pulling of the one or more elastically deformable wires 256 as the balloon 230 is pulled into the carrier tube 260 provides additional radial compression on the balloon 230, as compared to an embodiment without the one or more elastically deformable wires 256 present, so that at least a portion of the drug remaining in the balloon 230 exits through the plurality of holes 232 of the balloon 230.

In other embodiments, the drug eluting apparatus 200 includes the carrier tube 260 and the ring 270, but does not include the one or more elastically deformable wires 256. The balloon 230 may be inflated with the drug to enter the radially expanded state 234a so that the drug exits through the plurality of holes 232 of the balloon 230 to enter the vessel. As the actuator 208 is actuated to pull the shaft 210 and radially compress the balloon 230 into the radially compressed state 234b, the balloon 230 is pulled into the carrier tube 260. Pulling the balloon 230 into the carrier tube 260 radially compresses the balloon 230 so that at least a portion of the drug remaining in the balloon 230 exits through the plurality of holes 232 of the balloon. As the actuator 208 is actuated to pull the shaft 210 and radially compress the balloon 230 into the radially compressed state 234b, the balloon 230 is pulled into the carrier tube 260 and into the ring 270. Pulling the balloon 230 into the carrier tube 260 and into the ring 270 provides additional radial compression on the balloon 230, as compared to an embodiment without the ring 270 present, so that at least a portion of the drug remaining in the balloon 230 exits through the plurality of holes 232 of the balloon 230.

In FIGS. 11A-11B, in one embodiment, the drug eluting apparatus 200 includes the carrier tube 260, the one or more elastically deformable wires 256, and the ring 270. In FIGS. 11A-11B, the balloon 230 may be inflated with the drug to enter the radially expanded state 234a so that the drug exits through the plurality of holes 232 of the balloon 230 to enter the vessel. In FIG. 11B, as the actuator 208 is actuated to pull the shaft 210 and radially compress the balloon 230 into the radially compressed state 234b, the balloon 230 is pulled into the carrier tube 260 and into the ring 270. Actuating the actuator 208 to pull the shaft 210 and radially compress the balloon 230 into the radially compressed state 234b also compresses the one or more elastically deformable wires 256, which radially compresses the balloon 230. Pulling the balloon 230 into the carrier tube 260 and into the ring 270 together with pulling the one or more elastically deformable wires 256 provide additional radial compression on the balloon 230, as compared to an embodiment with only the ring 270 or only the one or more elastically deformable wires 256 present, so that the drug remaining in the balloon 230 exits through the plurality of holes 232 of the balloon 230.

### Third Embodiment

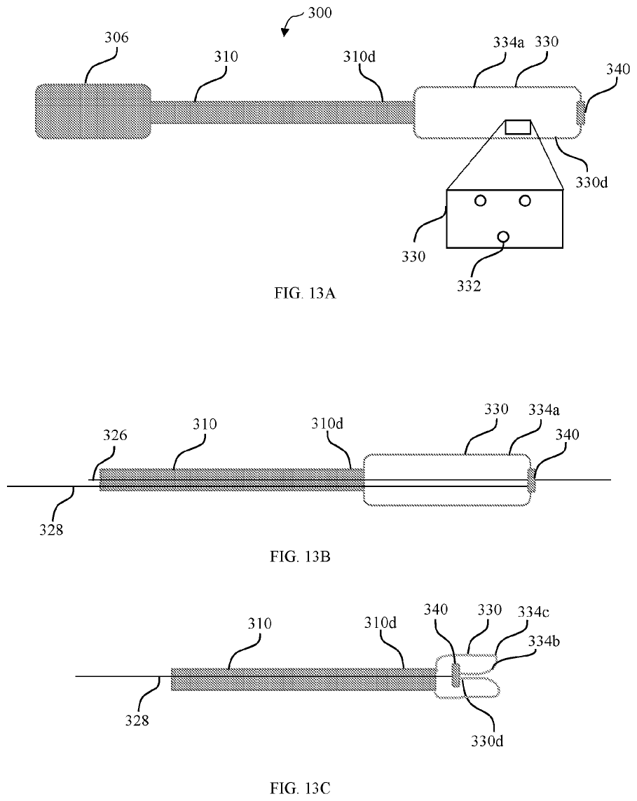

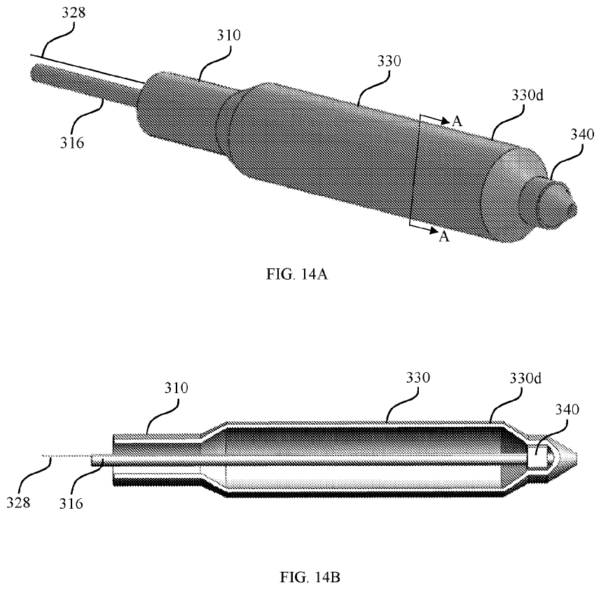

FIG. 13A-14B depict a drug eluting apparatus 300 (e.g., treatment apparatus, etc.) for delivering a drug (e.g., a drug that includes an anti-proliferative agent such as sirolimus, etc.) into a vessel according to a third embodiment. The drug eluting apparatus 300 includes a handle 306 having a drug port 304 configured to receive a drug. The drug eluting apparatus 300 further includes a shaft 310 connected to the handle 306. The shaft 310 includes a drug lumen 314 configured to receive the drug from the drug port 304, and a guidewire tube 316 configured to receive a guidewire 326 and allow the guidewire 326 to extend out of a distal portion 310d of the shaft 310. The drug eluting apparatus 300 further includes a balloon 330 attached to the distal portion 310d of the shaft 310 and configured to receive the drug from the drug lumen 314, the balloon 330 including a plurality of holes 332 configured to allow the drug to exit the balloon 330. The drug eluting apparatus 300 further includes an anchor 340 connected to a distal portion 330d of the balloon 330. The drug eluting apparatus 300 further includes a pull wire 328 connected to the anchor 340. The handle 306 includes an actuator 308 that is actuatable by a user to pull the pull wire 328 and thereby cause the balloon 330 to change from a longitudinally expanded state 334a to a longitudinally compressed state 334b. The longitudinally compressed state 334b of the balloon is an inverted state 334c. In some embodiments, the drug eluting apparatus 300 may further include a stopcock 302 connected to the drug port 304. In some embodiments, the shaft 310 may further include a pull wire lumen 318 and the pull wire 328 is located in the pull wire lumen 318 of the shaft 310.

The drug eluting apparatus 300 includes a handle 306 (e.g., grip, handgrip, etc.). The handle 306 is configured in the same way as the handle 106 of FIG. 2A-2B. The handle 306 includes a body 307 (e.g. shell, housing, container, etc.) that is configured in the same way as the body 107. The handle 306 also includes a hub 301 that is configured in the same way as the hub 101 of FIG. 2C. The hub 301 includes a drug port 304 (e.g., hole, opening, aperture, etc.) that is configured in the same way as the drug port 104. The handle 306 also includes an actuator 308 (e.g., tab, button, wheel, knob, etc.) that is configured in the same way as the actuator 108. The handle 306 may also include a stopcock 302 (e.g., valve, etc.) that is configured in the same way as the stopcock 102.

The drug eluting apparatus 300 also includes a shaft 310 (e.g., catheter, tube, pipe, cylinder, conduit, etc.) that is configured in the same way as the shaft 110.

The drug eluting apparatus 300 may be a rapid-exchange type device that is configured in the same way as the drug eluting apparatus 100, as in FIG. 3. In other embodiments, the drug eluting apparatus 300 may be an over-the-wire type device, in which the guidewire 326 enters the shaft 310 through the handle 306.

The shaft 310 includes a drug lumen 314 (e.g., cavity, space, etc.) that is configured in the same way as the drug lumen 114. In some embodiments, the shaft 310 also includes a pull wire lumen 318 (e.g., cavity, space, etc.) that is configured in the same way as the pull wire lumen 118. The shaft 310 also includes a guidewire tube 316 (e.g., pipe, cylinder, conduit, etc.) that is configured in the same way as the guidewire tube 116. The drug lumen 314, the pull wire lumen 318, and the guidewire tube 316 may be configured in the same way as the drug lumen 114, the pull wire lumen 118, and the guidewire tube 116 of FIGS. 4A-4C and FIGS. 5A-5C.

The drug eluting apparatus 300 also includes a balloon 330 that is configured in the same way as the balloon 130. The balloon 330 includes a plurality of holes 332 (e.g., apertures, openings, slits, etc.) that are configured in the same way as the plurality of holes 132 of the balloon 130.

The drug eluting apparatus 300 also includes an anchor 340 (e.g., block, weight, cap, etc.) that is configured in the same way as the anchor 140.

The drug eluting apparatus 300 also includes a pull wire 328 (e.g., thread, strand, line, filament, etc.) that is configured in the same way as the pull wire 128.

The pull wire 328 may be pulled by the actuator 308 when a user actuates the actuator 308. Pulling the pull wire 328 thereby causes the balloon 330 to change from a longitudinally expanded state 334a to a longitudinally compressed state 334b that is an inverted state 334c. In the inverted state 334c, the distal portion 330d of the balloon 330 is pulled proximally such that the distal portion 330d of the balloon changes from a concave shape to a convex shape. In FIG. 13B and FIG. 14A-14B, the balloon 330 is in a longitudinally expanded state 334a. In FIG. 13C, the balloon 330 is in a longitudinally compressed state 334b that is an inverted state 334c. As the balloon 330 changes from the longitudinally expanded state 334a to the longitudinally compressed state 334b that is an inverted state 334c, at least a portion of the drug that was remaining in the balloon 330, but that had not yet exited through the balloon 330, exits through the plurality of holes 332 of the balloon 330 and enters the vessel.

In FIG. 13A-13C, the drug eluting apparatus 300 includes an anchor 340 connected to a distal portion 330d of the balloon 330 and the anchor 340 is connected to a pull wire 328, and the longitudinally compressed state 334b of the balloon 330 is an inverted state 334c. The balloon 330 may be inflated with the drug to the longitudinally expanded state 334a in FIG. 13B. The actuator 308 may then be actuated to pull the pull wire 328 to pull the anchor 340 to compress the balloon 330 such that the balloon 330 enters the longitudinally compressed state 334b that is an inverted state 334c in FIG. 13C. When the balloon 330 transitions from the longitudinally expanded state 334a of FIG. 13B, to the longitudinally compressed state 334b and inverted state 334c of FIG. 13C, the distal portion 330d of the balloon 330 is pulled proximally via the anchor 340 such that the distal portion 330d of the balloon 330 moves proximally beyond another portion of the balloon 330. The balloon 330 may be made of a compliant material such that when the distal portion 330d of the balloon 330 is pulled by the pull wire 328 via the anchor 340, the balloon 330 enters the inverted state 334c of FIG. 13C instead of the longitudinally compressed state 134b of FIG. 1B. When the balloon 330 changes from the longitudinally expanded state 334a to the inverted state 334c, at least a portion of the drug remaining in the balloon 330 exits the balloon 330 through the plurality of holes 332.

In some embodiments, the drug eluting apparatus 300 may be used with a long carrier tube 180 (discussed in further detail herein). In other embodiments, the drug eluting apparatus 300 may be used with a short carrier tube 182 (discussed in further detail herein). In some embodiments, the drug eluting apparatus 300 may be used with the long carrier tube 180 or the short carrier tube 182, which is configured to be attached to a guiding catheter 184 (discussed in further detail herein).

### Fourth Embodiment

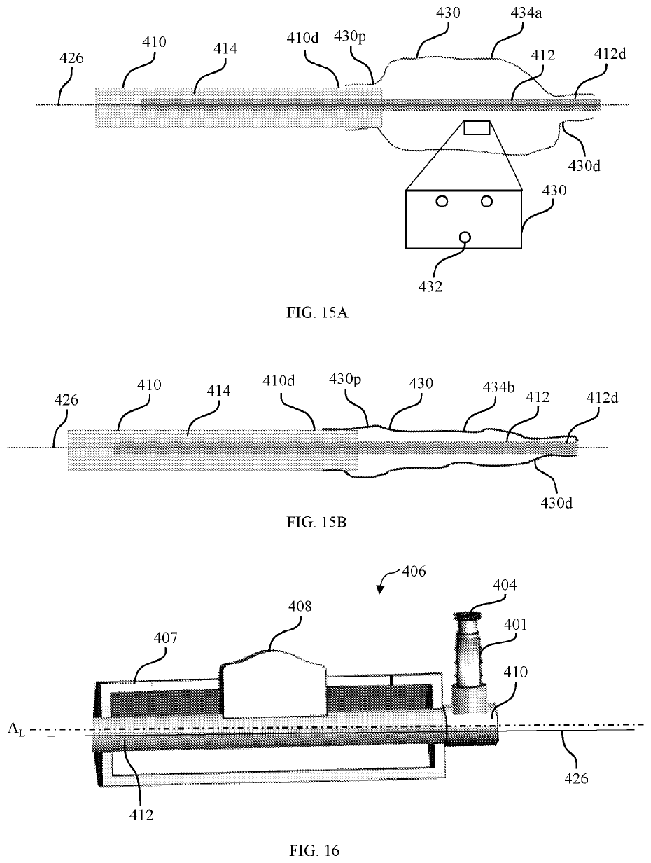

FIG. 15A-16 depict a drug eluting apparatus 400 (e.g., treatment apparatus, etc.) for delivering a drug (e.g., a drug that includes an anti-proliferative agent such as sirolimus, etc.) into a vessel according to a fourth embodiment. The drug eluting apparatus 400 includes a handle 406 having a drug port 404 configured to receive a drug. The drug eluting apparatus 400 further includes a first shaft 410 connected to the handle 406, the first shaft 410 including a drug lumen 414 further configured to receive the drug from the drug port 404. The drug eluting apparatus 400 further includes a second shaft 412 connected to the handle 406, the second shaft 412 configured to extend through and move independently of the first shaft 410. The second shaft 412 is configured to receive a guidewire 426 and allow the guidewire 426 to extend out of a distal portion 412d of the second shaft 412. The drug eluting apparatus 400 further includes a balloon 430 including a proximal portion 430p connected to a distal portion 410d of the first shaft 410, and a distal portion 430d connected to a distal portion 412d of the second shaft 412. The balloon 430 is configured to receive the drug from the drug lumen 414, and the balloon 430 has a plurality of holes 432 configured to allow the drug to exit the balloon 430. The actuator 408 is actuatable by a user to rotate the second shaft 412 relative to the first shaft 410 and thereby cause the balloon 430 to change from a radially expanded state 434a to a radially compressed state 434b. In some embodiments, the drug eluting apparatus 400 may further include a stopcock 402 connected to the drug port 404.

The drug eluting apparatus 400 includes a handle 406 (e.g., grip, handgrip, etc.). The handle 406 is configured to be held by a user and allows a user to operate the drug eluting apparatus 400. The handle 406 may be configured to be held with one hand, so as to allow a user to perform additional maneuvers with the user's second hand.

The handle 406 includes a body 407 (e.g. shell, housing, container, etc.) that is configured in the same way as the body 107.

The drug eluting apparatus 400 includes a hub 401. In FIG. 15, the hub 401 is located separate from the handle 406 and is connected to the first shaft 410. The hub 401 may be connected to the first shaft 410 by an adhesive or by other suitable connecting arrangements. However, in other embodiments, the hub 401 may be located at the top, bottom, left, right, or proximal side of the handle 406. The hub 401 includes a drug port 404 (e.g., hole, opening, aperture, etc.) that is configured in the same way as the drug port 104. The hub 401 may also include a stopcock 402 (e.g., valve, etc.) that is configured in the same way as the stopcock 102.

The handle 406 also includes an actuator 408 (e.g., tab, button, wheel, knob, etc.). The actuator 408 is a mechanical structure that is actuatable by a user to operate the drug eluting apparatus 400. The actuator 408 is actuated by a user of the drug eluting apparatus 400 by rotating the actuator 408 in either a clockwise or a counterclockwise direction about the length of the handle 406 in a direction parallel to the longitudinal axis A_{L}. Rotating the actuator 408 results in rotating the second shaft 412 relative to the first shaft 410 to radially compress the balloon 430 and cause the drug to exit the plurality of holes 432 of the balloon 430. In FIG. 15, the actuator 408 is located at a top side of the handle 406. However, in other embodiments, the actuator 408 may be located at a bottom, left, or right side of the handle 406.

The drug eluting apparatus 400 also includes a first shaft 410 (e.g., catheter, tube, pipe, cylinder, conduit, etc.). The first shaft 410 is connected to the handle 406. The first shaft 410 may be connected to the handle 406 by bonding with an adhesive, or by other suitable connecting arrangements. The first shaft 410 may be permanently fixed to the handle 406 so that the user may not disconnect the first shaft 410 from the handle 406.

The drug eluting apparatus 400 may be an over-the-wire type device, in which the guidewire 426 enters the second shaft 412 through the handle 406, as in FIG. 16. In other embodiments, the drug eluting apparatus 400 may be a rapid-exchange type device.

The first shaft 410 may be made of a fluorine polymer (e.g., polytetrafluoroethylene (PTFE)), ethylene/tetrafluoroethylene copolymer (ETFE), polyetheretherketone (PEEK), polyurethane, polyamide elastomer, polyester elastomer, polyurethane elastomer, polyimide, polycarbonate, polyetherimide, fluorocarbon resin, or other suitable materials.

The first shaft 410 may have a length that is approximately in a range of 30-300 cm, for example, 30-200 cm. The first shaft 410 may have a cross-sectional shape that is a circle, an oval, a polygon, a rounded polygon, or other geometric shape. The first shaft 410 may have a maximum cross-sectional width (e.g., a diameter where the cross-sectional shape is circular) that is approximately in a range of 1-15 mm, inclusive, and for example 2-8 mm.

The first shaft 410 is configured to receive the drug from the drug port 404. The first shaft 410 has a drug lumen 414 (e.g., cavity, space, etc.). The drug lumen 414 extends longitudinally through the first shaft 410. The drug lumen 414 is configured to receive the drug from the drug port 404.

The drug lumen 414 may have a cross-sectional shape that is a circle, an oval, a polygon, a rounded polygon, or other geometric shape. The drug lumen 414 may have a maximum cross-sectional width (e.g., a diameter where the cross-sectional shape is circular) that is approximately in a range of 0.5-10 mm, inclusive, and ideally 0.5-7 mm.

The drug eluting apparatus 400 also includes a second shaft 412 (e.g., catheter, tube, pipe, cylinder, conduit, etc.). The second shaft 412 is connected to the actuator 408. The second shaft 412 may be connected to the actuator 408 by bonding with an adhesive, by an interlocking feature, by welding, by press fit, by threaded fastener, or by other suitable connecting arrangements. The second shaft 412 may be permanently fixed to the actuator 408 so that the user may not disconnect the second shaft 412 from the actuator 408. A portion of the second shaft 412 is positioned within the handle 406 such that the second shaft 412 may be moved in a direction parallel to and may be rotated about a longitudinal axis A_{L} of the first shaft 410.

The second shaft 412 is configured to extend through and move independently of the first shaft 410. The second shaft 412 is configured such that the distal portion 412d of the second shaft 412 may be positioned distal to the distal portion 410d of the first shaft 410. The second shaft 412 is configured to receive a guidewire 426 and allow the guidewire 426 to extend out of a distal portion 412d of the second shaft 412.

The second shaft 412 may be made of a fluorine polymer (e.g., polytetrafluoroethylene (PTFE)), ethylene/tetrafluoroethylene copolymer (ETFE), polyetheretherketone (PEEK), polyurethane, polyamide elastomer, polyester elastomer, polyurethane elastomer, polyimide, polycarbonate, polyetherimide, fluorocarbon resin, or other suitable materials.

The second shaft 412 may have a length that is approximately in a range of 30-300 cm, for example, 30-200 cm. The second shaft 412 may have a cross-sectional shape that is a circle, an oval, a polygon, a rounded polygon, or other geometric shape. The second shaft 412 may have a maximum cross-sectional width (e.g., a diameter where the cross-sectional shape is circular) that is approximately in a range of 0.1-10 mm, inclusive, and for example, 0.5-3 mm.

The drug eluting apparatus 400 also includes a balloon 430. The balloon 430 has a proximal portion 430p that is attached to the distal portion 410d of the first shaft 410. The balloon 430 has a distal portion 430d that is attached to the distal portion 412d of the second shaft 412. The balloon 430 may be attached to the distal portion 410d of the first shaft 410 and may be attached to the distal portion 412d of the second shaft 412 by being placed over the distal portion 410d of the first shaft 410 and over the distal portion 412d of the second shaft 412 and bonded with an adhesive, or by other suitable connecting arrangements. In some embodiments, the proximal portion 430p of the balloon 430 may be attached to the distal portion 410d of the first shaft 410 in the same connecting arrangement as the distal portion 430d of the balloon 430 may be attached to the distal portion 412d of the second shaft 412. In other embodiments, the proximal portion 430p of the balloon 430 may be attached to the distal portion 410d of the first shaft 410 in a different connecting arrangement as the distal portion 430d of the balloon 430 may be attached to the distal portion 412d of the second shaft 412.

The balloon 430 is configured to receive the drug from the drug lumen 414 of the first shaft 410. When the drug enters the balloon 430 from the drug lumen 414, the balloon 430 expands to a radially expanded state 434a and the drug exits the balloon 430 through the plurality of holes 432 of the balloon 430.

The balloon 430 may be made of polyamide, polyester, polyurethane, polyamide elastomer, polyester elastomer, polyurethane elastomer, or other suitable materials.

The balloon 430 may have a length that is approximately in a range of 10-300 mm, for example, 10-50 mm, or 15-40 mm. The balloon 430 may have a width that is approximately in a range of 1-8 mm, for example, 2-5 mm, or 4-8 mm.

The balloon 430 includes a plurality of holes 432 (e.g., apertures, openings, slits, etc.) that are configured to allow the drug to exit the balloon 430. The plurality of holes 432 of the balloon 430 are configured in the same way as the plurality of holes 132 of the balloon 130. When the balloon 430 is in the radially expanded state 434a, the drug passes through the plurality of holes 432 of the balloon 430 to exit the balloon 430 and enter the vessel.

The actuator 408 may be rotated when a user actuates the actuator 408. Rotating the actuator 408 causes the distal portion 412d of the second shaft 412 to rotate. As the second shaft 412 rotates relative to the first shaft 410, the first shaft 410 may be held in a fixed position. The second shaft 412 may rotate in a clockwise or a counterclockwise direction. As the second shaft 412 rotates relative to the first shaft 410, the balloon 430 changes from a radially expanded state 434a to a radially compressed state 434b. In FIG. 15A, the balloon 430 is in the radially expanded state 434a. In FIG. 15B, the balloon 430 is in the radially compressed state 434b. As the balloon 430 is radially compressed from the radially expanded state 434a to the radially compressed state 434b, at least a portion of the drug that was remaining in the balloon 430, but that had not yet exited the balloon 430, exits through the plurality of holes 432 of the balloon 430 and enters the vessel.

In some embodiments, the drug eluting apparatus 400 may be used with a long carrier tube 180 (discussed in further detail herein). In other embodiments, the drug eluting apparatus 400 may be used with a short carrier tube 182 (discussed in further detail herein). In some embodiments, the drug eluting apparatus 400 may be used with the long carrier tube 180 or the short carrier tube 182, which is configured to be attached to a guiding catheter 184 (discussed in further detail herein).

### General Operation of Drug Eluting Apparatuses, and Long and Short Carrier Tubes

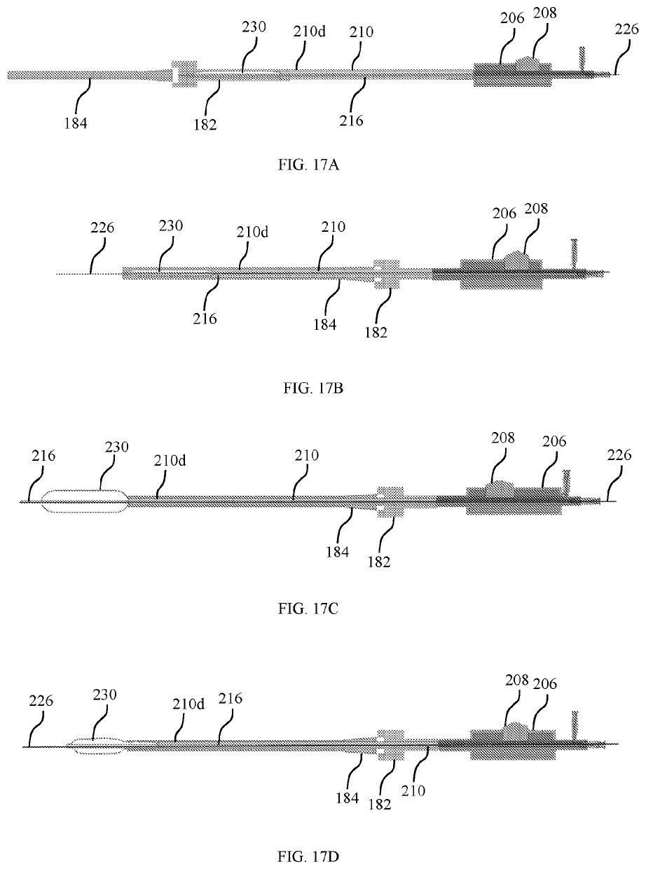

FIGS. 17A-19D depict drug eluting apparatuses in different configurations of operation of the drug eluting apparatuses. The drug eluting apparatuses may be used with a needle, a trocar, a guiding catheter, a short carrier tube, a long carrier tube, an inserter, a valve, and/or a flushing device.

The drug eluting apparatuses 100, 200, 300, and 400 may be used with a needle for insertion into a subject. The needle may be used to create a hole in the subject that provides access to a vessel. The vessel that the needle provides access to may be the vessel that is the target of drug elution or may be a vessel through which the drug eluting apparatus 100, 200, 300, and 400 may be pushed to reach another vessel that is the target of drug elution. In other embodiments, a trocar may be used to create the hole in the subject that provides access to the vessel.

After the needle has provided access to a vessel, a guidewire, which may be the guidewire 126, 226, 326, or 426 may be pushed through the needle to enter the vessel and positioned at or near the target of drug elution. Imaging may be used to facilitate positioning the guidewire in the subject. The needle is then withdrawn from the subject, and the guidewire is maintained in the subject. The guidewire is used to insert a portion of the drug eluting apparatus 100, 200, 300, or 400 into the subject. The guidewire may then be withdrawn from the vessel, and the components passed over the guidewire are maintained in the vessel. In other embodiments, the guidewire may remain in the vessel, and after the drug eluting apparatus 100, 200, 300, or 400 is used and withdrawn, the guidewire is withdrawn.

The drug eluting apparatuses 100, 200, 300, and 400 may be used with a guiding catheter 184 (e.g., catheter, shaft, tube, pipe, cylinder, conduit, etc.). The guiding catheter 184 is passed over a guidewire and inserted into the vessel. The guiding catheter 184 may be positioned at or near the target of drug elution. The shaft 110 and balloon 130, the shaft 210 and the balloon 230, the shaft 310 and the balloon 330, or the first shaft 410, second shaft 412, and balloon 430 may be passed through the guiding catheter 184.

Use of a guiding catheter 184 allows the balloon 130, 230, 330, or 430 to advance through the vessel without encountering calcifications, plaques, or other materials in the vessel that could interrupt the advancement of the balloon 130, 230, 330, or 430 if otherwise encountered in the absence of a guiding catheter 184. When a guiding catheter 184 is used, pulling the balloon 130, 230, 330, or 430 into the guiding catheter 184 radially compresses the balloon, as the balloon is pulled through the guiding catheter 184 and the guiding catheter 184 is distal to the long carrier tube 180 or the short carrier tube 182.

FIGS. 17A-19D depict drug eluting apparatuses with long carrier tubes 180 (e.g., a sheath, guiding sheath, cover, etc.) or short carrier tubes 182 (e.g., a sheath, guiding sheath, cover, etc.). FIGS. 17A-19D depict the drug eluting apparatus 200. In other embodiments, the drug eluting apparatus 100, 300, or 400 may be used as the drug eluting apparatus in FIGS. 17A-19D. In some embodiments, a long carrier tube 180, which may be the carrier tube 260, has a length that is equal to or greater than a length of a shaft, which may be the shaft 210. In other embodiments, a long carrier tube 180, which may be used with a drug eluting apparatus 100, 300, or 400, has a length that is equal to or greater than a length of a shaft, which may be the shaft 110, shaft 210, or first shaft 410. In some embodiments, a short carrier tube 182, which may be the carrier tube 260, has a length that is in a range of approximately 100-120% of the length of the balloon 230, and preferably 110%. In other embodiments, a short carrier tube 182, which may be used with a drug eluting apparatus 100, 300, or 400, has a length that is in a range of approximately 100-120% of the length of the balloon, which may be the balloon 130, 330, or 430, and preferably 110%. Long carrier tubes 180 and short carrier tubes 182 may be used with the guiding catheter 184.

FIGS. 17A-17D depict a drug eluting apparatus with a short carrier tube 182 and a guiding catheter 184. The short carrier tube 182 may connect with the guiding catheter 184 such that a shaft 110, 210, 310, or first shaft 410 and second shaft 412 may pass first through the short carrier tube 182 and second through the guiding catheter 184. The short carrier tube 182 may connect with the guiding catheter 184 by snap fit, by a Luer lock connector, or by other suitable connecting arrangements. The short carrier tube 182 may connect to a handle, which may be the handle 106, 206, 306, or 406, by a Luer lock connector, a lock connector, a fitting connector, or by other suitable connecting arrangements. In the configuration of FIG. 17A, the short carrier tube 182 is not connected to the handle 206. In the configurations of FIG. 17B-17D, the short carrier tube 182 is connected to the handle 206.

Use of a short carrier tube 182 and a guiding catheter 184 allows for protection of the balloon, which may be the balloon 130, 230, 330, or 430, during insertion of the balloon and allows the balloon to be primed and then directly inserted into the guiding catheter 184, which eliminates the introduction of air in the guiding catheter 184. Use of a short carrier tube 182 facilitates transfer of the balloon to the guiding catheter 184.

In FIGS. 17A-17D, the short carrier tube 182 is shown with the drug eluting apparatus 200. In FIG. 17A, the balloon 230 and the shaft 210 are positioned in the short carrier tube 182. In FIG. 17B, the balloon 230 and the shaft 210 have been pushed through the short carrier tube 182 and into the guiding catheter 184. When the balloon 230 is in the guiding catheter 184, the balloon 230 is in the deformed state of the radially compressed state 234b. In FIG. 17C, the balloon 230 and the shaft 210 have been pushed forward such that the balloon 230 is fully outside of the guiding catheter 184. In FIG. 17C, the balloon 230 is inflated, and the balloon 230 is in the radially expanded state 234a. After the drug has been delivered, the balloon 230 and the shaft 210 may be pulled back such that the balloon 230 is inside of the guiding catheter 184, as in FIG. 17B, or in the short carrier tube 182, as in FIG. 17A. In FIG. 17D, the balloon 230 and the shaft 210 are being pulled back such that the balloon 230 is partially inside of the guiding catheter 184. Pulling back the shaft 210 radially compresses the balloon 230 as the balloon 230 is compressed against the guiding catheter 184.

In other embodiments, the short carrier tube 182 of FIGS. 17A-17D, may be used with the drug eluting apparatus 100, 300, or 400. FIGS. 17A-17D depict an over-the-wire type device. In other embodiments, the short carrier tube 182 of FIGS. 17A-17D may be used with a rapid-exchange type device. When the short carrier tube 182 is used with a rapid-exchange type device and the guiding catheter 184, the guidewire 126, 226, 336, or 436 is positioned alongside the shaft 110, 210, 310, or first shaft 410 and second shaft 412 and inside the guiding catheter 184.

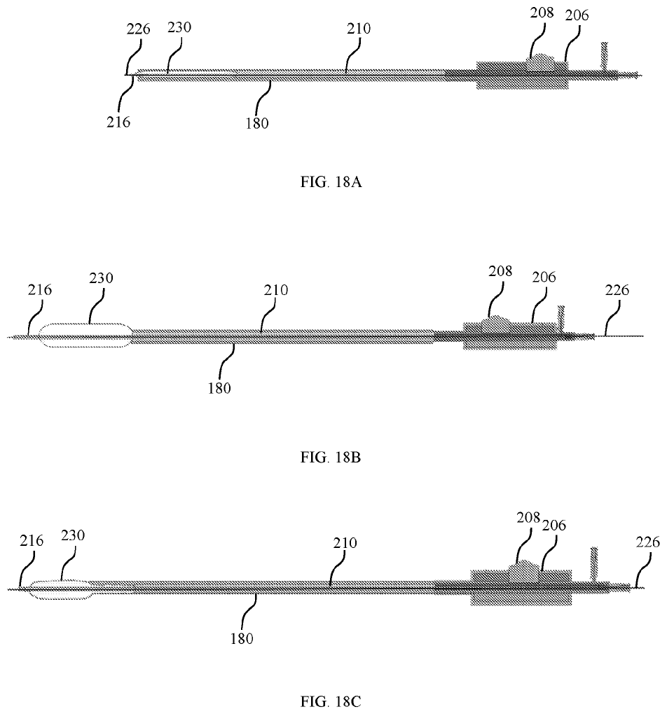

In FIG. 18A-18C, the long carrier tube 180 is shown with the drug eluting apparatus 200. The long carrier tube 180 connects to a handle 206 and is connected by a Luer lock connector, a lock connector, a fitting connector, or by other suitable connecting arrangements. In FIG. 18A, the balloon 230 is positioned in the long carrier tube 180In FIG. 18B, the balloon 230 has been pushed forward such that the balloon 230 is fully outside of the long carrier tube 180. In FIG. 18B, the balloon 230 is inflated and is in the radially expanded state 234a. In FIG. 18C, the balloon 230 is being pulled back such that the balloon 230 is partially inside of the long carrier tube 180. After the drug has been delivered, the balloon 230 may be pulled back such that the balloon 230 is inside of the long carrier tube 180, as in FIG. 18A.

In other embodiments, the long carrier tube 180 of FIG. 18A-18C may be used with the drug eluting apparatus 100, 300, or 400. In some embodiments, the long carrier tube 180 of FIG. 18A-18C may be used with the guiding catheter 184 of FIGS. 17A-17D in place of the short carrier tube 182. FIG. 18A-18C depict an over-the-wire type device. In other embodiments, the long carrier tube 180 of FIG. 18A-18C may be used with a rapid-exchange type device. When the long carrier tube 180 is used with a rapid-exchange type device and the guiding catheter 184, the guidewire 126, 226, 336, or 436 is positioned alongside the shaft 110, 210, 310, or first shaft 410 and second shaft 412, and is positioned inside the guiding catheter 184.

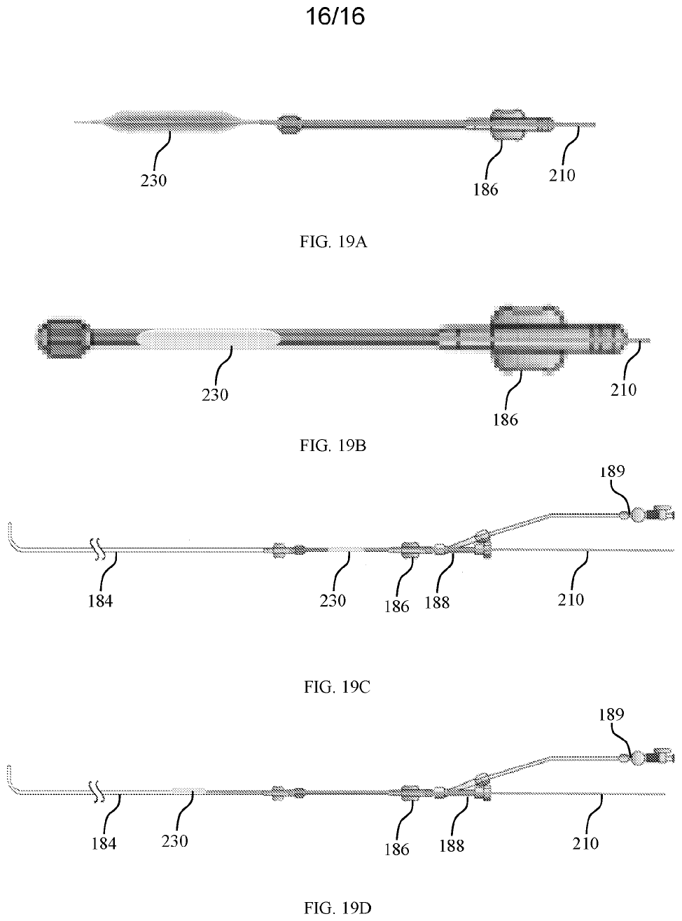

FIGS. 19A-19D depict a portion of a drug eluting apparatus with the guiding catheter 184, an inserter 186 (e.g., shaft, tube, pipe, cylinder, conduit, etc.), a valve 188 (e.g., a connection, a gate, a tap, etc.), and a flushing device 189. In FIGS. 19A-19D, the drug eluting apparatus is the drug eluting apparatus 200. In other embodiments, the drug eluting apparatus of FIGS. 19A-19D may be the drug eluting apparatus 100, 300, or 400. The guiding catheter 184 may be connected to the inserter 186 by a Luer lock connector or by other suitable connecting arrangements.

The inserter 186 may be connected to the valve 188 by a Luer lock connector or by other suitable connecting arrangements. The inserter 186 may be passed over the guidewire 226. The use of an inserter 186 to provide the balloon 230 to the guiding catheter 184 may facilitate the pushing of the balloon 230 in the guiding catheter 184 as without an inserter 186 the balloon 230 may buckle when pushed or pushing the balloon 230 may introduce air into the guiding catheter 184.

In FIG. 19C-19D, the valve 188 is a three-way valve configured to connect to the inserter 186, to connect to a flushing device 189, and to receive a portion of the drug eluting apparatus 200, such as the shaft 210. In other embodiments, the valve 188 may be a two-way valve configured to connect to the inserter 186 and to receive a portion of the drug eluting apparatus 200, such as the shaft 210. The valve 188 may be passed over the guidewire 226.

The flushing device 189 may be used to flush the inserter 186 and/or the guiding catheter 184 so that the inserter 186 is de-aired, which allows for an easier and safer transition of the balloon 230 into the guiding catheter 184 without air.

In FIGS. 19A-19D, the guiding catheter 184, an inserter 186, and a valve 188 are shown with a portion of the drug eluting apparatus 200. In FIG. 19A, the shaft 210 is pulled through the inserter 186. In FIG. 19B, the shaft 210 is pulled through the inserter 186 such that the balloon 230 is in the inserter 186. In FIG. 19C, the shaft 210 is pulled such that the shaft 210 passes through the valve 188. In FIG. 19C, the inserter 186 is shown as connected to the guiding catheter 184 and also connected to the valve 188. Before or after use of the inserter 186, guiding catheter 184, and valve 188, the inserter 186 may be unconnected to the guiding catheter 184 and also unconnected to the valve 188. The inserter 186 may be connected to the guiding catheter 184 by a Luer lock connector, or by other suitable connecting arrangements. The inserter 186 may be connected to the valve 188 by a Luer lock connector, or by other suitable connecting arrangements. In FIG. 19D, the shaft 210 is pushed such that the balloon 230 and the shaft 210 enter the guiding catheter 184.

In other embodiments, the guiding catheter 184, the inserter 186, the valve 188, and/or the flushing device 189 of FIGS. 19A-19D may be used with the drug eluting apparatus 100, 300, or 400. In other embodiments, the inserter 186, the valve 188, and/or the flushing device 189 of FIGS. 19A-19D may be used with the long carrier tube 180 or the short carrier tube 182. In some embodiments, the inserter 186, the valve 188, and/or the flushing device 189 may be used with a rapid-exchange type device. In other embodiments, the inserter 186, the valve 188, and/or the flushing device 189 may be used with an over-the-wire type device.

### Methods of Delivering Drugs Using Drug Eluting Apparatuses

An example first method for delivering a drug into a vessel includes providing a drug eluting apparatus 100. The first method also includes inserting the distal portion 110d of the shaft 110 into the vessel. The first method also includes providing the drug to the balloon 130 via the drug lumen 114 so as to inflate the balloon 130 and deliver the drug into the vessel via the plurality of holes 132 of the balloon 130. The first method also includes compressing the balloon 130 by using the actuator 108 to pull the pull wire 128, thereby causing the balloon 130 to change from the longitudinally expanded state 134a to the longitudinally compressed state 134b and expelling at least a portion of the drug remaining in the balloon 130. The first method also includes allowing the elastically deformable wire 150 to cause the balloon 130 to return to the longitudinally expanded state 134a.

The first method includes providing a drug eluting apparatus 100. The drug eluting apparatus 100 includes a handle 106 having a drug port 104 configured to receive a drug. The drug eluting apparatus 100 further includes a shaft 110 connected to the handle 106. The shaft 110 has a drug lumen 114 configured to receive the drug from the drug port 104, and a guidewire tube 116 configured to receive a guidewire 126 and allow the guidewire 126 to extend out of a distal portion 110d of the shaft 110. The drug eluting apparatus 100 further includes a balloon 130 attached to the distal portion 110d of the shaft 110 and configured to receive the drug from the drug lumen 114, the balloon 130 having a plurality of holes 132 to allow the drug to exit the balloon 130. The drug eluting apparatus 100 further includes an anchor 140 connected to a distal portion of the balloon 130. The drug eluting apparatus 100 further includes an elastically deformable wire 150 having a distal end 150d connected to the distal portion 130d of the balloon 130, and a proximal end 150p connected to the guidewire tube 116. The drug eluting apparatus 100 further includes a pull wire 128 connected to the anchor 140. The handle 106 further includes an actuator 108 that is actuatable by a user to pull the pull wire 128 and thereby cause the balloon 130 to change from a longitudinally expanded state 134a to a longitudinally compressed state 134b. After the pull wire 128 is pulled and the balloon 130 is compressed, the elastically deformable wire 150 causes the balloon 130 to return to the longitudinally expanded state 134a. In some embodiments, the drug eluting apparatus 100 may further include a stopcock 102 connected to the drug port 104. In some embodiments, the shaft 110 may further include a pull wire lumen 118 and the pull wire 128 is located in the pull wire lumen 118 of the shaft 110. While the first method is described with respect to the drug eluting apparatus 100, it should be appreciated that the operations of the first method are equally applicable to any other apparatus that includes components analogous to those described therein.

The first method includes inserting the distal portion 110d of the shaft 110 into the vessel, such that the distal portion 110d of the shaft 110 is at a location in the vessel that is a target of drug elution. Inserting the distal portion 110d of the shaft 110 into the vessel may include using a needle, a trocar, a guiding catheter 184 as in FIGS. 17A-17D, an inserter 186 as in FIGS. 19A-19D, a valve 188 as in FIGS. 19A-19D, and/or a flushing device 189 as in FIGS. 19A-19D. The drug eluting apparatus 100 may be used with the short carrier tube 182 of FIGS. 17A-17D or the long carrier tube 180 of FIG. 18A-18C.

The first method includes providing the drug to the balloon 130 via the drug lumen 114 so as to inflate the balloon 130 and deliver the drug into the vessel via the plurality of holes 132 of the balloon 130. When the balloon 130 is inflated, the balloon 130 is in the longitudinally expanded state 134a, as in FIG. 1A. Pressure to the balloon 130 in its longitudinally expanded state 134a is maintained by the drug entering the balloon 130.

The first method includes compressing the balloon 130 by using the actuator 108 to pull the pull wire 128, thereby causing the balloon 130 to change from the longitudinally expanded state 134a of FIG. 1A to the longitudinally compressed state 134b of FIG. 1B and expelling at least a portion of the drug remaining in the balloon 130. The pull wire 128 pulls on the anchor 140 to longitudinally compress the balloon 130.

The first method includes allowing the elastically deformable wire 150 to cause the balloon 130 to return to the longitudinally expanded state 134a, as in FIG. 1C. The actuator 108 may cease being actuated such that the pull wire 128 ceases pulling the pull wire 128, allowing the balloon 130 to recover its length as it returns to the longitudinally expanded state 134a. The elastically deformable wire 150 may expand to support the longitudinally expanded state 134a of the balloon 130.

An example second method for delivering a drug into a vessel includes providing a drug eluting apparatus 200 using the long carrier tube 180 as the carrier tube 260. The second method also includes inserting the distal portion 210d of the shaft 210 and the distal portion 260d of the carrier tube 260 into the vessel. The second method also includes pushing the shaft 210 or pulling the carrier tube 260 so that the distal portion 210d of the shaft 210 and the balloon 230 are outside of the carrier tube 260. The second method also includes providing the drug to the balloon 230 via the drug lumen 214 so as to inflate the balloon 230 and deliver the drug into the vessel via the plurality of holes 232 of the balloon 230. The second method also includes compressing the balloon 230 by using the actuator 208 to pull the shaft 210, thereby pulling the balloon 230 into the carrier tube 260, causing the balloon 230 to change from a radially expanded state 234a to a radially compressed state 234b, and expelling at least a portion of the drug remaining in the balloon 230.

The second method includes providing a drug eluting apparatus 200. The drug eluting apparatus 200 includes a handle 206 having a drug port 204 configured to receive a drug. The drug eluting apparatus 200 further includes a shaft 210 connected to the handle 206. The shaft 210 has a drug lumen 214 configured to receive the drug from the drug port 204, and a guidewire tube 216 configured to receive a guidewire 226 and allow the guidewire 226 to extend out of a distal portion 210d of the shaft 210. The drug eluting apparatus 200 further includes a carrier tube 260 connected to the handle 206, the carrier tube 260 surrounding the shaft 210. The carrier tube 260 has a length that is equal to or greater than a length of the shaft 210. The drug eluting apparatus 200 further includes a balloon 230 attached to a distal portion 210d of the shaft 210 and configured to receive the drug from the drug lumen 214, the balloon 230 including a plurality of holes 232 configured to allow the drug to exit the balloon 230. The handle 206 includes an actuator 208 that is actuatable by a user to pull the shaft 210 and thereby pull the balloon 230 into the carrier tube 260 and cause the balloon 230 to change from a radially expanded state 234a to a radially compressed state 234b. In some embodiments, the drug eluting apparatus 200 may further include a stopcock 202 connected to the drug port 204. In some embodiments, the drug eluting apparatus 200 may further include one or more elastically deformable wires 256 that extend from a proximal portion 230p of the balloon 230 to a distal portion 230d of the balloon 230 and are curved radially outward relative to a longitudinal axis A_{L} of the shaft 210 when in a relaxed state. In some embodiments, the drug eluting apparatus 200 may further include a ring 270 connected to a distal portion 210d of the carrier tube 260. An elastic modulus of a material of the ring 270 is greater than an elastic modulus of a material of the carrier tube 260. While the second method is described with respect to the drug eluting apparatus 200, it should be appreciated that the operations of the second method are equally applicable to any other apparatus that includes components analogous to those described therein.

The second method includes inserting the distal portion 210d of the shaft 210 and the distal portion 260d of the carrier tube 260 into the vessel, such that the distal portion 210d of the shaft 210 is at a location in the vessel that is a target of drug elution. In some embodiments, during the insertion the distal portion 210d of the shaft 210 may be positioned proximal to the distal portion 260d of the carrier tube 260, as in FIG. 8A. In other embodiments, during the insertion the distal portion 260d of the carrier tube 260 may be positioned distal to the distal portion 210d of the shaft 210, as in FIG. 8B.

Inserting the distal portion 210d of the shaft 210 into the vessel may include using a needle, a trocar, a guiding catheter 184 as in FIGS. 17A-17D, an inserter 186 as in FIGS. 19A-19D, a valve 188 as in FIGS. 19A-19D, and/or a flushing device 189 as in FIGS. 19A-19D.

The second method includes pushing the shaft 210 or pulling the carrier tube 260 so that the distal portion 210d of the shaft 210 and the balloon 230 are outside of the carrier tube 260. When the distal portion 210d of the shaft 210 is proximal to the distal portion 260d of the carrier tube 260, the shaft 210 is pushed forward or the carrier tube 260 is pulled so that the distal portion 210d of the shaft 210 is distal to the distal portion 260d of the carrier tube 260.

The second method includes providing the drug to the balloon 230 via the drug lumen 214 so as to inflate the balloon 230 and deliver the drug into the vessel via the plurality of holes 232 of the balloon 230. When the balloon 230 is inflated, the balloon 230 is in the radially expanded state 234a, as in FIG. 8C. Pressure to the balloon 230 in its radially expanded state 234a is maintained by the drug entering the balloon 230.

The second method includes compressing the balloon 230 by using the actuator 208 to pull the shaft 210, thereby causing the balloon 230 to change from the radially expanded state 234a of FIG. 8C to the radially compressed state 234b of FIG. 8D and expelling at least a portion of the drug remaining in the balloon 230. Pulling the shaft 210 radially compresses the balloon 230 into the radially compressed state 234b as the balloon 230 is pulled into the carrier tube 260.

In some embodiments, the drug eluting apparatus 200 includes the one or more elastically deformable wires 256. When the actuator 208 is used to pull the shaft 210 to pull the balloon 230 into the carrier tube 260, the one more elastically deformable wires 256 provide additional compression of the balloon 230, as compared to an embodiment without the one or more elastically deformable wires 256 present. In some embodiments, the drug eluting apparatus 200 includes the ring 270. When the actuator 208 is used to pull the shaft 210 to pull the balloon 230 into the carrier tube 260, the balloon 230 is pulled into the ring 270 to provide additional compression of the balloon 230, as compared to an embodiment without the ring 270 present. In some embodiments, the drug eluting apparatus 200 includes the one or more elastically deformable wires 256 and the ring 270. When the actuator 208 is used to pull the shaft 210 to pull the balloon 230 into the carrier tube 260, the one more elastically deformable wires 256 and the pulling of the balloon 230 into the ring 270 provide additional compression of the balloon 230, as compared to an embodiment with only the one more elastically deformable wires 256 or only the ring 270 present.

In some embodiments, the second method includes inserting a guiding catheter 184 into the vessel before inserting the distal portion 210d of the shaft 210 and the distal portion 260d of the carrier tube 260 into the vessel. The carrier tube 260 is connected to the guiding catheter 284. In the step of inserting the distal portion 210d of the shaft 210 and the distal portion 260d of the carrier tube 260 into the vessel, the distal portion 210d of the shaft 210 is inserted into the guiding catheter 284. In the step of pushing the shaft 210 or pulling the carrier tube 260, the shaft 210 is pushed so that the distal portion 210d of the shaft 210 and the balloon 230 are outside of the guiding catheter 184. In the step of compressing the balloon 230, the balloon 230 is pulled into the guiding catheter 184 so that the balloon 230 is radially compressed into the radially compressed state 234b as the balloon 230 is pulled into the guiding catheter 184.

An example third method for delivering a drug into a vessel includes providing a drug eluting apparatus 200 using the short carrier tube 182 as the carrier tube 260. The third method also includes inserting the distal portion 210d of the shaft 210, the distal portion 260d of the carrier tube 260, and a guiding catheter 184 into the vessel. The third method also includes pushing the shaft 210 so that the distal portion 210d of the shaft 210 and the balloon 230 are outside of the carrier tube 260 and the guiding catheter 184. The third method also includes providing the drug to the balloon 230 via the drug lumen 214 so as to inflate the balloon 230 and deliver the drug into the vessel via the plurality of holes 232 of the balloon 230. The third method also includes compressing the balloon 230 by using the actuator 208 to pull the shaft 210, thereby pulling the balloon 230 into the carrier tube 260, causing the balloon 230 to change from a radially expanded state 234a to a radially compressed state 234b, and expelling at least a portion of the drug remaining in the balloon 230.

The third method includes providing a drug eluting apparatus 200. The drug eluting apparatus 200 includes a handle 206 having a drug port 204 configured to receive a drug. The drug eluting apparatus 200 further includes a shaft 210 connected to the handle 206. The shaft 210 has a drug lumen 214 configured to receive the drug from the drug port 204, and a guidewire tube 216 configured to receive a guidewire 226 and allow the guidewire 226 to extend out of a distal portion 210d of the shaft 210. The drug eluting apparatus 200 further includes a carrier tube 260 connected to or connectable to the handle 206, the carrier tube 260 surrounding the shaft 210. The carrier tube 260 has a length that is less than a length of the shaft 210. The drug eluting apparatus 200 further includes a balloon 230 attached to a distal portion 210d of the shaft 210 and configured to receive the drug from the drug lumen 214, the balloon 230 including a plurality of holes 232 configured to allow the drug to exit the balloon 230. The handle 206 includes an actuator 208 that is actuatable by a user to pull the shaft 210 and thereby pull the balloon 230 into the carrier tube 260 and cause the balloon 230 to change from a radially expanded state 234a to a radially compressed state 234b. In some embodiments, the drug eluting apparatus 200 may further include a stopcock 202 connected to the drug port 204. In some embodiments, the drug eluting apparatus 200 may further include one or more elastically deformable wires 256 that extend from a proximal portion 230p of the balloon 230 to a distal portion 230d of the balloon 230 and are curved radially outward relative to a longitudinal axis A_{L} of the shaft 210 when in a relaxed state. In some embodiments, the drug eluting apparatus 200 may further include a ring 270 connected to a distal portion 210d of the carrier tube 260. An elastic modulus of a material of the ring 270 is greater than an elastic modulus of a material of the carrier tube 260. While the third method is described with respect to the drug eluting apparatus 200, it should be appreciated that the operations of the third method are equally applicable to any other apparatus that includes components analogous to those described therein.

The third method includes inserting the distal portion 210d of the shaft 210, the distal portion 260d of the carrier tube 260, and the guiding catheter 184 into the vessel, such that the distal portion 210d of the shaft 210 is at a location in the vessel that is a target of drug elution. In some embodiments, during the insertion the distal portion 210d of the shaft 210 may be positioned in the short carrier tube 182, as in FIG. 17A. In other embodiments, during the insertion the distal portion 260d of the carrier tube 260 may be positioned in the guiding catheter 184, as in FIG. 17B.

Inserting the distal portion 210d of the shaft 210 into the vessel may include using a needle, a trocar, an inserter 186 as in FIGS. 19A-19D, a valve 188 as in FIGS. 19A-19D, and/or a flushing device 189 as in FIGS. 19A-19D.

The third method includes pushing the shaft 210 so that the distal portion 210d of the shaft 210 and the balloon 230 are outside of the carrier tube 260 and the guiding catheter 184. When the distal portion 210d of the shaft 210 is positioned in the short carrier tube 182, the shaft 210 is pushed forward so that the distal portion 210d of the shaft 210 is in the guiding catheter 184. When the distal portion 210d of the shaft 210 is positioned in the guiding catheter 184, the shaft 210 is pushed forward so that the distal portion 210d of the shaft 210 is outside of the guiding catheter 184.

The third method includes providing the drug to the balloon 230 via the drug lumen 214 so as to inflate the balloon 230 and deliver the drug into the vessel via the plurality of holes 232 of the balloon 230. When the balloon 230 is inflated, the balloon 230 is in the radially expanded state 234a, as in FIG. 8C. Pressure to the balloon 230 in its radially expanded state 234a is maintained by the drug entering the balloon 230.

The third method includes compressing the balloon 230 by using the actuator 208 to pull the shaft 210, thereby causing the balloon 230 to change from the radially expanded state 234a of FIG. 8C to the radially compressed state 234b of FIG. 8D and expelling at least a portion of the drug remaining in the balloon 230. Pulling the shaft 210 radially compresses the balloon 230 into the radially compressed state 234b as the balloon 230 is pulled into the guiding catheter 184.

In some embodiments, the drug eluting apparatus 200 includes the one or more elastically deformable wires 256. When the actuator 208 is used to pull the shaft 210 to pull the balloon 230 into the carrier tube 260, the one more elastically deformable wires 256 provide additional compression of the balloon 230, as compared to an embodiment without the one or more elastically deformable wires 256 present. In some embodiments, the drug eluting apparatus 200 includes the ring 270. When the actuator 208 is used to pull the shaft 210 to pull the balloon 230 into the carrier tube 260, the balloon 230 is pulled into the ring 270 to provide additional compression of the balloon 230, as compared to an embodiment without the ring 270 present. In some embodiments, the drug eluting apparatus 200 includes the one or more elastically deformable wires 256 and the ring 270. When the actuator 208 is used to pull the shaft 210 to pull the balloon 230 into the carrier tube 260, the one more elastically deformable wires 256 and the pulling of the balloon 230 into the ring 270 provide additional compression of the balloon 230, as compared to an embodiment with only the one more elastically deformable wires 256 or only the ring 270 present.

An example fourth method for delivering a drug into a vessel includes providing a drug eluting apparatus 300. The fourth method also includes inserting the distal portion 310d of the shaft 310 into the vessel. The fourth method also includes providing the drug to the balloon 330 via the drug lumen 334 so as to inflate the balloon 330 and deliver the drug into the vessel via the plurality of holes 332 of the balloon 330. The fourth method also includes compressing the balloon 330 by using the actuator 308 to pull the pull wire 328, thereby causing the balloon 330 to change from the longitudinally expanded state 334a to the longitudinally compressed state 334b, the longitudinally compressed state 334b of the balloon 330 being an inverted state 334c, and expelling at least a portion of the drug remaining in the balloon 330.

The fourth method includes providing a drug eluting apparatus 300. The drug eluting apparatus 300 includes a handle 306 having a drug port 304 configured to receive a drug. The drug eluting apparatus 300 further includes a shaft 310 connected to the handle 306. The shaft 310 includes a drug lumen 314 configured to receive the drug from the drug port 304, and a guidewire tube 316 configured to receive a guidewire 326 and allow the guidewire 326 to extend out of a distal portion 310d of the shaft 310. The drug eluting apparatus 300 further includes a balloon 330 attached to the distal portion 310d of the shaft 310 and configured to receive the drug from the drug lumen 314, the balloon 330 including a plurality of holes 332 configured to allow the drug to exit the balloon 330. The drug eluting apparatus 300 further includes an anchor 340 connected to a distal portion 330d of the balloon 330. The drug eluting apparatus 300 further includes a pull wire 328 connected to the anchor 340. The handle 306 includes an actuator 308 that is actuatable by a user to pull the pull wire 328 and thereby cause the balloon 330 to change from a longitudinally expanded state 334a to a longitudinally compressed state 334b. The longitudinally compressed state 334b of the balloon is an inverted state 334c. In some embodiments, the drug eluting apparatus 300 may further include a stopcock 302 connected to the drug port 304. In some embodiments, the shaft 310 may further include a pull wire lumen 318 and the pull wire 328 is located in the pull wire lumen 318 of the shaft 310. While the fourth method is described with respect to the drug eluting apparatus 300, it should be appreciated that the operations of the fourth method are equally applicable to any other apparatus that includes components analogous to those described therein.

The fourth method includes inserting the distal portion 310d of the shaft 310 into the vessel, such that the distal portion 310d of the shaft 310 is at a location in the vessel that is a target of drug elution. Inserting the distal portion 310d of the shaft 310 into the vessel may include using a needle, a trocar, a guiding catheter 184 as in FIGS. 17A-17D, an inserter 186 as in FIGS. 19A-19D, a valve 188 as in FIGS. 19A-19D, and/or a flushing device 189 as in FIGS. 19A-19D. The drug eluting apparatus 300 may be used with the short carrier tube 182 of FIGS. 17A-17D or the long carrier tube 180 of FIG. 18A-18C.

The fourth method includes providing the drug to the balloon 330 via the drug lumen 314 so as to inflate the balloon 330 and deliver the drug into the vessel via the plurality of holes 332 of the balloon 330. When the balloon 330 is inflated, the balloon 130 is in the longitudinally expanded state 134a, as in FIG. 13A-13B. Pressure to the balloon 330 in its longitudinally expanded state 334a is maintained by the drug entering the balloon 330.

The fourth method includes compressing the balloon 330 by using the actuator 308 to pull the pull wire 328, thereby causing the balloon 330 to change from the longitudinally expanded state 334a of FIG. 13A-13B to the longitudinally compressed state 334b that is the inverted state 334c of FIG. 13C and expelling at least a portion of the drug remaining in the balloon 330. The pull wire 328 pulls on the anchor 340 to invert and compress the balloon 330. The inversion of the balloon 330 compresses the balloon 330.

An example fifth method for delivering a drug into a vessel includes providing a drug eluting apparatus 400. The fifth method also includes inserting the distal portion 410d of the first shaft 410 and the distal portion 412d of the second shaft 412 into the vessel. The fifth method also includes providing the drug to the balloon 430 via the drug lumen 414 so as to inflate the balloon 430 and deliver the drug into the vessel via the plurality of holes 432 of the balloon 430. The fifth method also includes compressing the balloon 430 by using the actuator 408 to rotate the second shaft 412 relative to the first shaft 410, thereby causing the balloon 430 to change from the radially expanded state 434a to the radially compressed state 434b and expelling at least a portion of the drug remaining in the balloon 430.

The fifth method includes providing a drug eluting apparatus 400. The drug eluting apparatus 400 includes a handle 406 having a drug port 404 configured to receive a drug. The drug eluting apparatus 400 further includes a first shaft 410 connected to the handle 406, the first shaft 410 including a drug lumen 414 further configured to receive the drug from the drug port 404. The drug eluting apparatus 400 further includes a second shaft 412 connected to the handle 406, the second shaft 412 configured to extend through and move independently of the first shaft 410. The second shaft 412 is configured to receive a guidewire 426 and allow the guidewire 426 to extend out of a distal portion 412d of the second shaft 412. The drug eluting apparatus 400 further includes a balloon 430 including a proximal portion 430p connected to a distal portion 410d of the first shaft 410, and a distal portion 430d connected to a distal portion 412d of the second shaft 412. The balloon 430 is configured to receive the drug from the drug lumen 414, and the balloon 430 has a plurality of holes 432 configured to allow the drug to exit the balloon 430. The actuator 408 is actuatable by a user to rotate the second shaft 412 relative to the first shaft 410 and thereby cause the balloon 430 to change from a radially expanded state 434a to a radially compressed state 434b. In some embodiments, the drug eluting apparatus 400 may further include a stopcock 402 connected to the drug port 404. While the fifth method is described with respect to the drug eluting apparatus 400, it should be appreciated that the operations of the fifth method are equally applicable to any other apparatus that includes components analogous to those described therein.

The fifth method includes inserting the distal portion 410d of the first shaft 410 and the distal portion 412d of the second shaft 412 into the vessel, such that the distal portion 410d of the first shaft 410 and the distal portion 412d of the second shaft 412 are at a location in the vessel that is a target of drug elution.

Inserting the distal portion 410d of the first shaft 410 and the distal portion 412d of the second shaft 412 into the vessel may include using a needle, a trocar, a guiding catheter 184 as in FIGS. 17A-17D, an inserter 186 as in FIGS. 19A-19D, a valve 188 as in FIGS. 19A-19D, and/or a flushing device 189 as in FIGS. 19A-19D. The drug eluting apparatus 400 may be used with the short carrier tube 182 of FIGS. 17A-17D or the long carrier tube 180 of FIGS. 18A-18C.

The fifth method includes providing the drug to the balloon 430 via the drug lumen 414 so as to inflate the balloon 430 and deliver the drug into the vessel via the plurality of holes 432 of the balloon 430. When the balloon 430 is inflated, the balloon 430 is in the radially expanded state 434a, as in FIG. 15A. Pressure to the balloon 430 in its radially expanded state 434a is maintained by the drug entering the balloon 430.

The fifth method includes compressing the balloon 430 by using the actuator 408 to rotate the second shaft 412 relative to the first shaft 410, thereby causing the balloon 430 to change from the radially expanded state 434a of FIG. 15A to the radially compressed state 434b of FIG. 15B and expelling at least a portion of the drug remaining in the balloon 430. The second shaft 412 may rotate in a clockwise or a counterclockwise direction.

### Construction of Example Embodiments

It should be noted that the term “example” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Additionally, it should be understood that features from one embodiment disclosed herein may be combined with features of other embodiments disclosed herein as one of ordinary skill in the art would understand. Other substitutions, modifications, changes and omissions may also be made in the design, operating conditions and arrangement of the various exemplary embodiments without departing from the scope of the present invention.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

As utilized herein, the terms “substantially,” “generally,” “approximately,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the appended claims.

Also, the term “or” is used, in the context of a list of elements, in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, Z, X and Y, X and Z, Y and Z, or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

Additionally, the use of ranges of values (e.g., W1 to W2, etc.) herein are inclusive of their maximum values and minimum values (e.g., W1 to W2 includes W1 and includes W2, etc.), unless otherwise indicated. Furthermore, a range of values (e.g., W1 to W2, etc.) does not necessarily require the inclusion of intermediate values within the range of values (e.g., W1 to W2 can include only W1 and W2, etc.), unless otherwise indicated.

1. A drug eluting apparatus comprising:
a handle comprising a drug port configured to receive a drug;
a shaft connected to the handle, the shaft comprising:
a drug lumen configured to receive the drug from the drug port, and
a guidewire tube configured to receive a guidewire and allow the guidewire to extend out of a distal portion of the shaft;
a balloon attached to the distal portion of the shaft and configured to receive the drug from the drug lumen, the balloon comprising a plurality of holes configured to allow the drug to exit the balloon;
an anchor connected to a distal portion of the balloon;
an elastically deformable wire having a distal end connected to the distal portion of the balloon, and a proximal end connected to the guidewire tube; and
a pull wire connected to the anchor; wherein:
the handle comprises an actuator that is actuatable by a user to pull the pull wire and thereby cause the balloon to change from a longitudinally expanded state to a longitudinally compressed state; and
after the pull wire is pulled and the balloon is longitudinally compressed, the elastically deformable wire causes the balloon to return to the longitudinally expanded state.

2. The drug eluting apparatus of claim 1, wherein the distal end of the elastically deformable wire is attached to the distal portion of the balloon via the anchor.

3. The drug eluting apparatus of claim 1, wherein the elastically deformable wire has a helical shape.

4. The drug eluting apparatus of claim 1, wherein the elastically deformable wire has a straight shape.

5. The drug eluting apparatus of claim 1, wherein the elastically deformable wire is made of nitinol or stainless steel.

6. The drug eluting apparatus of claim 1, wherein a spring constant of the elastically deformable wire varies along a length of the elastically deformable wire.

7. The drug eluting apparatus of claim 1, wherein the drug lumen and guidewire tube are separate from each other.

8. The drug eluting apparatus of claim 1, wherein the guidewire tube extends through the drug lumen.

9. The drug eluting apparatus of claim 1, further comprising a stopcock connected to the drug port.

10. The drug eluting apparatus of claim 1, wherein the shaft further comprises a pull wire lumen and the pull wire is located in the pull wire lumen of the shaft.

11. A drug eluting apparatus comprising:
a handle comprising a drug port configured to receive a drug;
a shaft connected to the handle, the shaft comprising:
a drug lumen configured to receive the drug from the drug port, and
a guidewire tube configured to receive a guidewire and allow the guidewire to extend out of a distal portion of the shaft;
a carrier tube connected to or connectable to the handle, the carrier tube surrounding the shaft; and
a balloon attached to a distal portion of the shaft and configured to receive the drug from the drug lumen, the balloon comprising a plurality of holes configured to allow the drug to exit the balloon; wherein:
the handle comprises an actuator that is actuatable by a user to pull the shaft and thereby pull the balloon into the carrier tube and cause the balloon to change from a radially expanded state to a radially compressed state.

12. The drug eluting apparatus of claim 11, further comprising a stopcock connected to the drug port.

13. The drug eluting apparatus of claim 11, further comprising one or more elastically deformable wires that extend from a proximal portion of the balloon to a distal portion of the balloon and are curved radially outward relative to a longitudinal axis of the shaft when in a relaxed state.

14. The drug eluting apparatus of claim 13, wherein the one or more elastically deformable wires are made of nitinol or stainless steel.

15. The drug eluting apparatus of claim 11, further comprising a ring connected to a distal portion of the carrier tube, wherein an elastic modulus of a material of the ring is greater than an elastic modulus of a material of the carrier tube.

16. The drug eluting apparatus of claim 15, wherein the ring is made of a radiopaque material.

17. The drug eluting apparatus of claim 11, wherein the carrier tube is configured to be attached to a guiding catheter.

18. A drug eluting apparatus comprising:
a handle comprising a drug port configured to receive a drug;
a shaft connected to the handle, the shaft comprising:
a drug lumen configured to receive the drug from the drug port, and
a guidewire tube configured to receive a guidewire and allow the guidewire to extend out of a distal portion of the shaft;
a balloon attached to the distal portion of the shaft and configured to receive the drug from the drug lumen, the balloon comprising a plurality of holes configured to allow the drug to exit the balloon;
an anchor connected to a distal portion of the balloon; and
a pull wire connected to the anchor; wherein:
the handle comprises an actuator that is actuatable by a user to pull the pull wire and thereby cause the balloon to change from a longitudinally expanded state to a longitudinally compressed state, wherein the longitudinally compressed state of the balloon is an inverted state.

19. The drug eluting apparatus of claim 18, wherein the drug lumen and guidewire tube are separate from each other.

20. The drug eluting apparatus of claim 18, wherein the guidewire tube extends through the drug lumen.

21. The drug eluting apparatus of claim 18, further comprising a stopcock connected to the drug port.

22. The drug eluting apparatus of claim 18, wherein the shaft further comprises a pull wire lumen and the pull wire is located in the pull wire lumen of the shaft.

23. A drug eluting apparatus comprising:
a handle comprising a drug port configured to receive a drug;
a first shaft connected to the handle, the first shaft comprising a drug lumen configured to receive the drug from the drug port;
a second shaft connected to the handle, the second shaft configured to extend through and move independently of the first shaft, and the second shaft configured to receive a guidewire and allow the guidewire to extend out of a distal portion of the second shaft; and
a balloon comprising a proximal portion connected to a distal portion of the first shaft, and a distal portion connected to a distal portion of the second shaft, the balloon configured to receive the drug from the drug lumen, and the balloon comprising a plurality of holes configured to allow the drug to exit the balloon; wherein:
the handle comprises an actuator that is actuatable by a user to rotate the second shaft relative to the first shaft and thereby cause the balloon to change from a radially expanded state to a radially compressed state.

24. The drug eluting apparatus of claim 23, further comprising a stopcock connected to the drug port.

25-58. (canceled)
